SEQUENCE LISTING (15 sequences)

SEQ ID NO: 1 - protein, 14 aa, Artificial Sequence
  Synthetic construct
QCRPSKGRKRGFCW

SEQ ID NO: 2 - protein, 20 aa, Artificial Sequence
  Synthetic construct
QCRPSKGRKRGFCWAVDKYG

SEQ ID NO: 3 - protein, 28 aa, Artificial Sequence
  Synthetic construct
KKGFYKKKQCRPSKGRKRGFCWAVDKYG

SEQ ID NO: 4 - protein, 12 aa, Artificial Sequence
  Synthetic construct
CRPSKGRKRGFC

SEQ ID NO: 5 - protein, 20 aa, Artificial Sequence
  Synthetic construct
GFYKKKQCRPSKGRKRGFCW

SEQ ID NO: 6 - protein, 22 aa, Artificial Sequence
  Synthetic construct
KKGFYKKKQCRPSKGRKRGFCW

SEQ ID NO: 7 - protein, 26 aa, Artificial Sequence
  Synthetic construct
KKGFYKKKQCRPSKGRKRGFCWNGRK

SEQ ID NO: 8 - protein, 40 aa, Artificial Sequence
  Synthetic construct
KGFYKKKQCRPSKGRKRGFCWAQTRRRERRAEKQAQWKAA

SEQ ID NO: 9 - DNA, 55 bp, Artificial Sequence
  Synthetic construct
agtttggcacaatcaataactttttcagttattgattgtgccaaactcctgtctc

SEQ ID NO: 10 - DNA, 66 bp, Artificial Sequence
  Synthetic construct
gatcggcagtttggcacaatcaataactgaaaaagttattgattgtgccaaactgttttttggaag

SEQ ID NO: 11 - DNA, 66 bp, Artificial Sequence
  Synthetic construct
aattcttccaaaaaacagtttggcacaatcaataactttttcagttattgattgtgccaaactgcg

SEQ ID NO: 12 - protein, 27 aa, Artificial Sequence
  Synthetic construct
KKGFYKKKQCRPSKGRKRGFCWCVDKY

SEQ ID NO: 13 - protein, 18 aa, Artificial Sequence
  Synthetic construct
KKGHAKDSQRYKVDESQS

SEQ ID NO: 14 - protein, 9 aa, Artificial Sequence
  Synthetic construct
KKGFYKKKQ

SEQ ID NO: 15 - protein, 18 aa, Artificial Sequence
  Synthetic construct
KKGFYKKKQCRPSKGRKR

(19) United States
(12) Patent Application Publication
(10) Pub. No.: US 2005/0244422 A1
(43) Pub. Date: Nov. 3, 2005

(54) Methods for delivering MBD peptide-linked agent into cells under conditions of cellular stress

(76) Inventors: Desmond Mascarenhas, Los Altos Hills, CA (US); Baljit K. Singh, Sunnyvale, CA (US)

Correspondence Address: MORRISON & FOERSTER LLP, 755 PAGE MILL RD, PALO ALTO, CA, 94304-1018, US

(21) Appl. No.: 11/109,161

(22) Filed: Apr. 18, 2005

Related U.S. Application Data
(60) Provisional application No. 60/563,141, filed on Apr. 16, 2004.
(60) Provisional application No. 60/563,676, filed on Apr. 19, 2004.
(60) Provisional application No. 60/657,826, filed on Mar. 1, 2005.

(51) Int. Cl. A61K 48/00; A61K 39/385

(52) U.S. Cl. 424/185.1; 435/4; 514/44

(57) ABSTRACT

The present invention is related to methods of delivering MBD peptide-linked agents into live cells. The methods described herein comprise contacting MBD peptide-linked agents to live cells under a condition of cellular stress. The methods of the invention may be used for therapeutic or diagnostic purposes.

### CROSS-REFERENCE TO RELATED APPLICATIONS

This application claims the priority benefit of provisional patent applications U.S. Ser. Nos. 60/563,141, filed Apr. 16, 2004; 60/563,676, filed Apr. 19, 2004; and 60/657,826, filed Mar. 1, 2005; all of which are incorporated herein in their entirety by reference.

### TECHNICAL FIELD

The invention relates to the field of medical diagnostics and therapeutics, and more particularly to methods of identifying individuals who are likely to respond to treatment with certain therapeutic modalities. The invention also relates to methods of delivering MBD peptide-linked agents into live cells.

### BACKGROUND ART

The so-called diseases of western civilization (chronic conditions such as arthritis, asthma, osteoporosis, and atherosclerosis, other cardiovascular diseases, cancers of the breast, prostate and colon, metabolic syndrome-related conditions such as diabetes and PCOS, neurodegenerative conditions such as Parkinson's and Alzheimer's, and ophthalmic diseases such as macular degeneration) are now increasingly being viewed as secondary to chronic inflammatory conditions and adiposity. A direct link between adiposity and inflammation has recently been demonstrated. Macrophages, potent donors of pro-inflammatory signals, are nominally responsible for this link: Obesity is marked by macrophage accumulation in adipose tissue (Weisberg S P et al [2003] J. Clin Invest 112: 1796-1808) and chronic inflammation in fat plays a crucial role in the development of obesity-related insulin resistance (Xu H, et al [2003] J. Clin Invest. 112: 1821-1830). Inflammatory cytokine IL-18 is associated with PCOS, insulin resistance and adiposity (Escobar-Morreale H F, et al [2004] J. Clin Endo Metab 89: 806-811). Systemic inflammatory markers such as CRP are associated with unstable carotid plaque, specifically, the presence of macrophages in plaque, which is associated with instability can lead to the development of an ischemic event (Alvarez Garcia B et al [2003] J Vasc Surg 38: 1018-1024). There are documented cross-relationships between these risk factors. For example, there is higher than normal cardiovascular risk in patients with RA (Dessein P H et al [2002] Arthritis Res. 4: R5) and elevated C-peptide (insulin resistance) is associated with increased risk of colorectal cancer (Ma J et al [2004] J. Natl Cancer Inst 96:546-553) and breast cancer (Malin A. et al [2004] Cancer 100: 694-700).

The genesis of macrophage involvement with diseased tissues is not yet fully understood, though various theories postulating the “triggering” effect of some secondary challenge (such as viral infection) have been advanced. What is observed is vigorous crosstalk between macrophages, T-cells, and resident cell types at the sites of disease. For example, the direct relationship of macrophages to tumor progression has been documented. In many solid tumor types, the abundance of macrophages is correlated with prognosis (Lin E Y and Pollard J W [2004] Novartis Found Symp 256: 158-168). Reduced macrophage population levels are associated with prostate tumor progression (Yang G et al [2004] Cancer Res 64:2076-2082) and the “tumor-like behavior of rheumatoid synovium” has also been noted (Firestein G S [2003] Nature 423: 356-361). At sites of inflammation, macrophages elaborate cytokines such as interleukin-1-beta and interleukin-6.

A ubiquitous observation in chronic inflammatory stress is the up-regulation of heat shock proteins at the site of inflammation, followed by macrophage infiltration, oxidative stress and the elaboration of cytokines leading to stimulation of growth of local cell types. For example, this has been observed with unilateral obstructed kidneys, where the sequence results in tubulointerstitial fibrosis and is related to increases in HSP70 in human patients (Valles, P. et al [2003] Pediatr Nephrol. 18: 527-535). HSP70 is required for the survival of cancer cells (Nylandsted J et al [2000] Ann NY Acad Sci 926: 122-125). Eradication of gliblastoma, breast and colon xenografts by HSP70 depletion has been demonstrated (Nylansted J et al [2002] Cancer Res 62:7139-7142; Rashmi R et al [2004] Carcinogenesis 25: 179-187) and blocking HSF1 by expressing a dominant-negative mutant suppresses growth of a breast cancer cell line (Wang J H et al [2002] BBRC 290: 1454-1461). It is hypothesized that stress-induced extracellular HSP72 promotes immune responses and host defense systems. In vitro, rat macrophages are stimulated by HSP72, elevating NO, TNF-a, IL-1-beta and IL-6 (Campisi J et al [2003] Cell Stress Chaperones 8: 272-86). Significantly higher levels of (presumably secreted) HSP70 were found in the sera of patients with acute infection compared to healthy subjects and these levels correlated with levels of IL-6, TNF-alpha, IL-10 (Njemini R et al [2003] Scand. J. Immunol 58: 664-669). HSP70 is postulated to maintain the inflammatory state in asthma by stimulating pro-inflammatory cytokine production from macrophages (Harkins M S et al [2003] Ann Allergy Asthma Immunol 91: 567-574). In esophageal carcinoma, lymph node metastasis is associated with reduction in both macrophage populations and HSP70 expression (Noguchi T. et al [2003] Oncol. 10: 1161-1164). HSPs are a possible trigger for autoimmunity (Purcell A W et al [2003] Clin Exp Immunol. 132: 193-200). There is aberrant extracellular expression of HSP70 in rheumatoid joints (Martin C A et al [2003] J. Immunol 171: 5736-5742). Even heterologous HSPs can modulate macrophage behavior: H. pylori HSP60 mediates IL-6 production by macrophages in chronically inflamed gastric tissues (Gobert A P et al [2004] J. Biol. Chem 279: 245-250).

In addition to immunological stress, a variety of environmental conditions can trigger cellular stress programs. For example, heat shock (thermal stress), anoxia, high osmotic conditions, hyperglycemia, nutritional stress, endoplasmic reticulum (ER) stress and oxidative stress each can generate cellular responses, often involving the induction of stress proteins such as HSP70.

Familial mutations in parkin gene are associated with early-onset PD. Parkinson's disease (PD) is characterized by the selective degeneration of dopaminergic (DA) neurons in the substantia nigra pars compacta (SNpc). A combination of genetic and environmental factors contributes to such a specific loss, which is characterized by the accumulation of misfolded protein within dopaminergic neurons. Among the five PD-linked genes identified so far, parkin, a 52 kD protein-ubiquitin E3 ligase, appears to be the most prevalent genetic factor in PD. Mutations in parkin cause autosomal recessive juvenile parkinsonism (AR-JP). The current therapy for Parkinson's disease is aimed to replace the lost transmitter, dopamine. But the ultimate objective in neurodegenerative therapy is the functional restoration and/or cessation of progression of neuronal loss (Jiang H, et al [2004] Hum Mol Genet. 13 (16): 1745-54; Muqit M M, et al [2004] Hum Mol Genet. 13 (1): 117-135; Goldberg M S, et al [2003] J Biol. Chem. 278 (44): 43628-43635). Over-expressed parkin protein alleviates PD pathology in experimental systems. Recent molecular dissection of the genetic requirements for hypoxia, excitotoxicity and death in models of Alzheimer disease, polyglutamine-expansion disorders, Parkinson disease and more, is providing mechanistic insights into neurotoxicity and suggesting new therapeutic interventions. An emerging theme is that neuronal crises of distinct origins might converge to disrupt common cellular functions, such as protein folding and turnover (Driscoll M, and Gerstbrein B. [2003] Nat Rev Genet. 4(3): 181-194). In PC12 cells, neuronally differentiated by nerve growth factor, parkin overproduction protected against cell death mediated by ceramide Protection was abrogated by the proteasome inhibitor epoxomicin and disease-causing variants, indicating that it was mediated by the E3 ubiquitin ligase activity of parkin. (Darios F. et al [2003] Hum Mol Genet. 12 (5): 517-526). Overexpressed parkin suppresses toxicity induced by mutant (A53T) and wt alpha-synuclein in SHSY-5Y cells (Oluwatosin-Chigbu Y. et al [2003] Biochem Biophys Res Commun. 309 (3): 679-684) and also reverses synucleinopathies in invertebrates (Haywood A F and Staveley B E. [2004] BMC Neurosci. 5(1): 14) and rodents (Yamada M, Mizuno Y, Mochizuki H. (2005) Parkin gene therapy for alpha-synucleinopathy: a rat model of Parkinson's disease. Hum Gene Ther. 16(2): 262-270; Lo Bianco C. et al [2004] Proc Natl Acad Sci USA. 101(50): 17510-17515). On the other hand, a recent report claims that parkin-deficient mice are not themselves a robust model for the disease (Perez F A and Palmiter R D [2005] Proc Natl Acad Sci USA. 102 (6): 2174-2179). Nevertheless, parkin therapy has been suggested for PD (Butcher J. [2005] Lancet Neurol. 4(2): 82).

Variability within patient populations creates numerous problems for medical treatment. Without reliable means for determining which individuals will respond to a given treatment, physicians are forced to resort to trial and error. Because not all patients will respond to a given therapy, the trial and error approach means that some portion of the patients must suffer the side effects (as well as the economic costs) of a treatment that is not effective in that patient.

For some therapeutics targeted to specific molecules within the body, screening to determine eligibility for the treatment is routinely performed. For example, the estrogen antagonist tamoxifen targets the estrogen receptor, so it is normal practice to only administer tamoxifen to those patients whose tumors express the estrogen receptor. Likewise, the anti-tumor agent trastuzumab (HERCEPTIN®) acts by binding to a cell surface molecule known as HER2/neu; patients with HER2/neu negative tumors are not normally eligible for treatment with trastuzumab. Methods for predicting whether a patient will respond to treatment with IGF-I/IGFBP-3 complex have also been disclosed (U.S. Pat. No. 5,824,467), as well as methods for creating predictive models of responsiveness to a particular treatment (U.S. Pat. No. 6,087,090).

IGFBP-3 is a master regulator of cellular function and viability. As the primary carrier of IGFs in the circulation, it plays a central role in sequestering, delivering and releasing IGFs to target tissues in response to physiological parameters such as nutrition, trauma, and pregnancy. IGFs, in turn, modulate cell growth, survival and differentiation, Additionally, IGFBP-3 can sensitize selected target cells to apoptosis in an IGF-independent manner. The mechanisms by which it accomplishes the latter class of effects is not well understood but appears to involve selective cell internalization mechanisms and vesicular transport to specific cellular compartments (such as the nucleus, where it may interact with transcriptional elements) that is at least partially dependent on transferrin receptor, integrins and caveolin.

The inventor has previously disclosed certain IGFBP-derived peptides known as “MBD” peptides (U.S. patent application publication nos. 2003/0059430, 2003/0161829, and 2003/0224990). These peptides have a number of properties, which are distinct from the IGF-binding properties of IGFBPs, that make them useful as therapeutic agents. MBD peptides are internalized some cells, and the peptides can be used as cell internalization signals to direct the uptake of molecules joined to the MBD peptides (such as proteins fused to the MBD peptide).

All references cited herein, including patent applications and publications, are incorporated by reference in their entirety.

### DISCLOSURE OF THE INVENTION

The present invention provides a method for delivering an MBD peptide-linked agent into live cells, said method comprising contacting said MBD peptide-linked agent to live cells that are under a condition of cellular stress, whereby said contact results in cellular uptake of said MBD-peptide-linked agent.

The invention also provides a method for obtaining diagnostic information from live cells comprising the steps of: (a) administering an MBD peptide-linked agent to live cells that are under a condition of cellular stress; (b) delivering said MBD peptide-linked agent into said live cells, whereby said agent creates a diagnostic readout that can be measured; and (c) measuring the diagnostic readout. The diagnostic readout can be an enzymatic, a calorimetric, or a fluorimetric readout.

The invention also provides a method for modifying in a disease process or a cellular process, said method comprising the steps of: (a) administering an MBD peptide-linked agent to live cells that are under a condition of cellular stress, wherein the agent is capable of modifying the disease process or the cellular process within said live cells; and (b) delivering said MBD peptide-linked agent into said live cells, whereby said disease process or said cellular process in said live cells is modified. In some embodiments, the disease process is selected from the group consisting of neurodegenerative, cancer, autoimmune, inflammatory, cardiovascular, diabetes, osteoporosis and ophthalmic diseases. In some embodiments, the cellular process is selected from the group consisting of transcriptional, translational, protein folding, protein degradation and protein phosphorylation events.

In some embodiments, the condition of cellular stress is selected from the group consisting of thermal, immunological, cytokine, oxidative, metabolic, anoxic, endoplasmic reticulum, protein unfolding, nutritional, chemical, mechanical, osmotic and glycemic stress. In some embodiments, the condition of cellular stress is associated with upregulation of at least about 1.5-fold of at least one of the genes shown in FIG. 7. In some embodiments, at least two, at least three, at least four, at least five, at least ten, at least fifteen, at least seventeen, or all of the genes shown in FIG. 7 are upregulated at least about 1.5-fold in the live cells under the condition of cellular stress compared to same type of live cells not under the condition of cellular stress. In some embodiments, the one or more genes are upregulated at least about 2-fold, at least about 3-fold, at least about 4-fold, at least about 5-fold, or at least about 10-fold under the condition of cellular stress.

In some embodiments, the methods described herein further comprise a step or steps for identifying the cells for delivering the MBD peptide-linked agent into the cells. Such steps may include comparing levels of gene expression of one or more of the genes shown in FIG. 7 in cells under the condition of cellular stress to levels of gene expression in the same type of cells not under the condition of cellular stress, and selecting cells that have at least one, at least two, at least three, at least four, at least five, at least ten, at least fifteen, at least seventeen, or all of the genes shown in FIG. 7 upregulated at least about 1.5-fold under the condition of cellular stress for delivering the MBD peptide-linked agent into the cells. In some embodiments, the one or more genes are upregulated at least about 2-fold, at least about 3-fold, at least about 4-fold, at least about 5-fold, or at least about 10-fold under the condition of cellular stress.

The agent linked to the MBD peptide may be a diagnostic agent or a therapeutic agent. In some embodiments, the agent is a protein or a peptide. In some embodiments, the agent is a nucleic acid. In some embodiments, the agent is a small molecule.

In some embodiments, the live cells are in a subject, such as a mammal. For example, the live cells are in a human. In some embodiments, the live cells are in a tissue or in cell culture.

Any MBD peptide described in U.S. Patent Application Nos. 2003/0059430, 2003/0161829, and 2003/0224990 (which are incorporated herein by reference in their entirety) may be used. In some embodiments, the MBD peptide comprises the amino acid sequence QCRPSKGRKRGFCW. In some embodiments, the MBD peptide comprises the amino acid sequence QCRPSKGRKRGFCW and a caveolin consensus binding sequence. In some embodiments, the MBD peptide comprises the amino acid sequence QCRPSKGRKRGFCWAVDKYG or KKGFYKKKQCRPSKGRKRGFCWAVDKYG.

The invention provides methods for identifying individuals who are candidates for treatment with MBD peptide-based therapies. MBD peptide-based therapies have been previously described in U.S. patent application publication nos. 2003/0059430, 2003/0161829, and 2003/0224990. However, the inventor has noted that there is variability in cellular internalization of MBD peptides. The invention provides methods for identifying which patients would be candidates for treatment with MBD peptide-based therapies, by predicting whether the relevant tissue(s) in the individual will take up MBD peptides.

In this invention I show that the physiological cellular state for which up-regulation of HSPs is emblematic is also the preferred state recognized by the MBD for cellular uptake and nuclear localization. MBD-mediated transport of appropriate macromolecules into cell nuclei at the sites of disease could allow for fine-tuned control of the disease process and for the design of very specific interventions. The possibility of delivery to sites of injury is also attractive. Liver injury leads to transcription of HSPs (Schiaffonati L and Tiberio L [1997] Liver. 17: 183-191) as does ischemia in isolated hearts (Nitta-Komatsubara Y et al [2000] 66:1261-1270). HSF1 is cardioprotective for ischemia/reperfusion injury (Zou Y et al [2003] Circulation 108: 3024-3030). This invention also provides for treatment of disorders characterized by secreted HSP70 and macrophage co-localized at the site of disease.

Privileged sites in the body also up-regulate HSPs constitutively, though most other cell types only induce HSPs as a specific response to stress. HSFs are required for spermatogenesis (Wang G et al [2004] Genesis 38: 66-80). Neuronal cells also display altered regulation of HSPs (Kaarniranta K et al [2002] Mol Brain Res 101:136-140). Longevity in C. elegans is regulated by HSF and chaperones (Morley J F and Morimoto R. I. [2004] Mol Biol Cell 15:657-664). MBD-mediated transport of regulatory macromolecules to such sites offers opportunities for interventions in neuroprotection and reproductive biology.

It is interesting that Kupffer cells (macrophage-like) are the major site of synthesis of IGFBP-3 in the liver (Scharf J et al [1996] Hepatology 23: 818-827; Zimmermann E M et al [2000] Am J. Physiol. Gastro. Liver Phys. 278: G447-457). Exogenously administered radiolabelled IGFBP-3 selectively accumulates in rat liver Kupffer cells (Arany E et al [1996] Growth Regul 6:32-41). Our earlier work suggested that caveolin and transferrin receptor were implicated in MBD-mediated cellular uptake. Caveolin is expressed in macrophages (Kiss A L et al [2002] Micron. 33: 75-93). Macrophage caveolin-1 is up-regulated in response to apoptotic stressors (Gargalovic P and Dory L [2003] J Lipid Res 44: 1622-1632). Macrophages express transferrin receptor (Mulero V and Brock J H [1999] Blood 94:2383-2389).

We are interested in elucidating the physiological and biochemical correlates of cellular receptivity to IGFBP-3, uptake and intracellular localization. We have recently localized and characterized the minimal sequence determinants of cellular recognition, uptake and intracellular localization to a C-terminal metal-binding domain (MBD) in the IGFBP-3 molecule. This domain, when to covalently linked to unrelated protein molecules such as GFP, can mediate specific cellular uptake and intracellular localization of such markers in selected cell systems. As a surrogate for the homing mechanism of IGFBP-3 itself, MBD-linked marker proteins can serve to elucidate patterns of cellular receptivity that might be otherwise be difficult or impossible to discern against a background of endogenous IGFBP-3.

Heat shock proteins (HSP) are molecular chaperones, involved in many cellular functions such as protein folding, transport, maturation and degradation. Since they control the quality of newly synthesized proteins, HSP take part in cellular homeostasis. The Hsp70 family in particular exerts these functions in an adenosine triphosphate (ATP)-dependent manner. ATP is the main energy source used by cells to assume fundamental functions (respiration, proliferation, differentiation, apoptosis). Therefore, ATP levels have to be adapted to the requirements of the cells and ATP generation must constantly compensate ATP consumption. Nevertheless, under particular stress conditions, ATP levels decrease, threatening cell homeostasis and integrity. Cells have developed adaptive and protective mechanisms, among which Hsp70 synthesis and over-expression is one.

Transferrin serves as the iron source for hemoglobin-synthesizing immature red blood cells. A cell surface receptor, transferrin receptor 1, is required for iron delivery from transferrin to cells. Transferrin receptor 1 has been established as a gatekeeper for regulating iron uptake by most cells. Iron uptake is viewed as an indicator of cellular oxidative metabolism and ATP-dependent metabolic rates.

In this study, we have dissected the molecular signatures of cells that selectively take up MBD-tagged markers.

By gene array and cellular protein analysis we have demonstrated that MBD-mediated protein uptake is linked to target cell physiological states resembling cellular responses to stress or injury. Thermal stress dramatically up-regulates uptake of MBD-tagged proteins. In vivo, inflammatory stress in an adjuvant arthritis rat model did not change the biodistribution of systemically administered MBD-tagged proteins. We are currently evaluating other in vivo and in vitro models of cellular stress.

### BRIEF DESCRIPTION OF THE DRAWINGS

FIG. 1 summarizes the results of the experiment described in Example 3.

FIG. 2 shows the IGFBP-3 metal-binding domain (MBD).

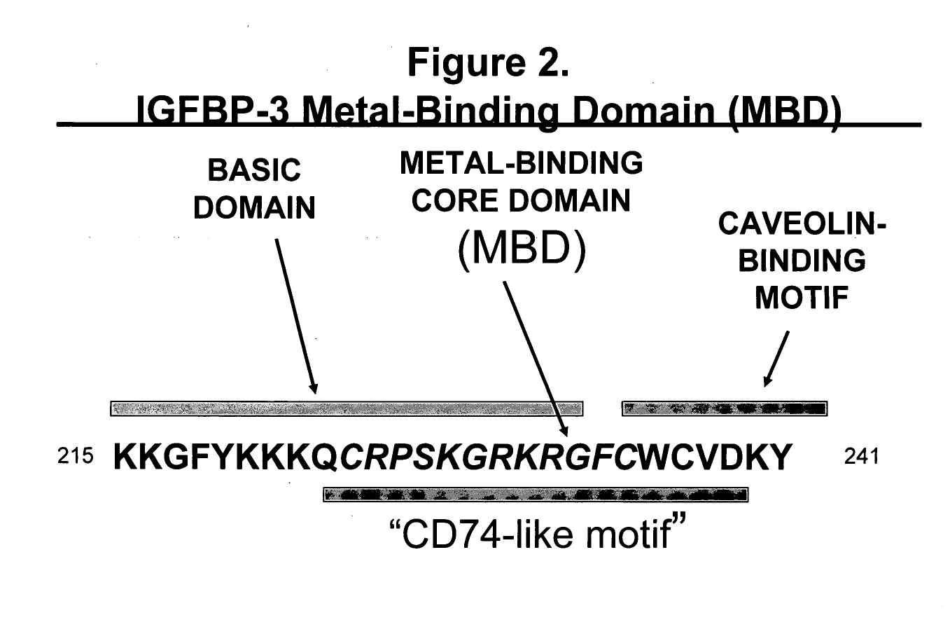

FIG. 3 shows the nuclear uptake of conjugate of various MBD and GFP.

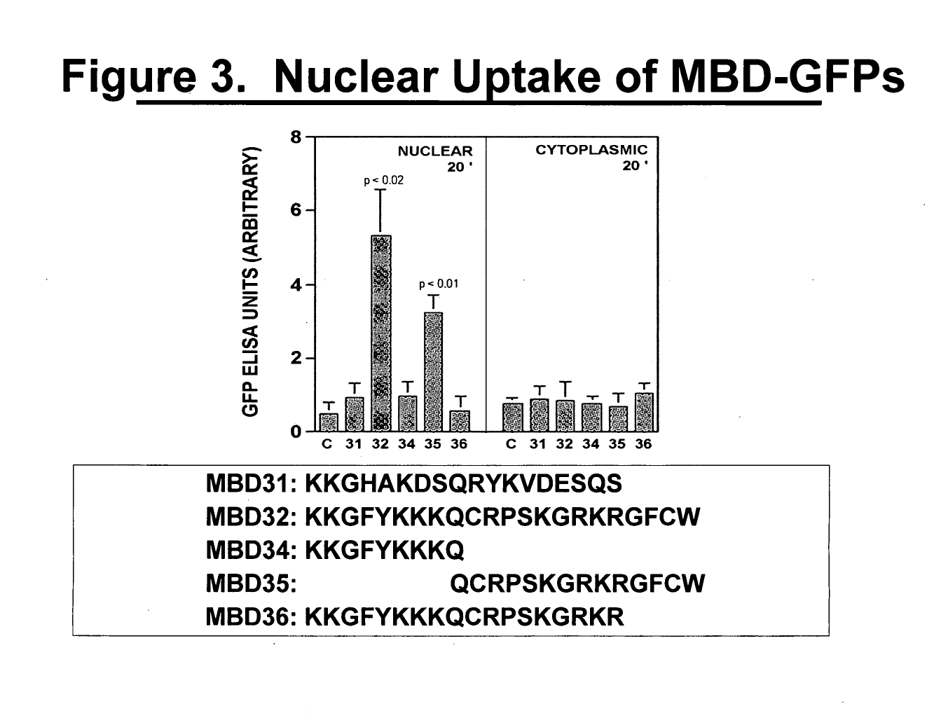

FIG. 4 shows the uptake of MBD-mobilized SA-HRP by tumor cell lines. A broad collection of anatomical sites was used in this survey.

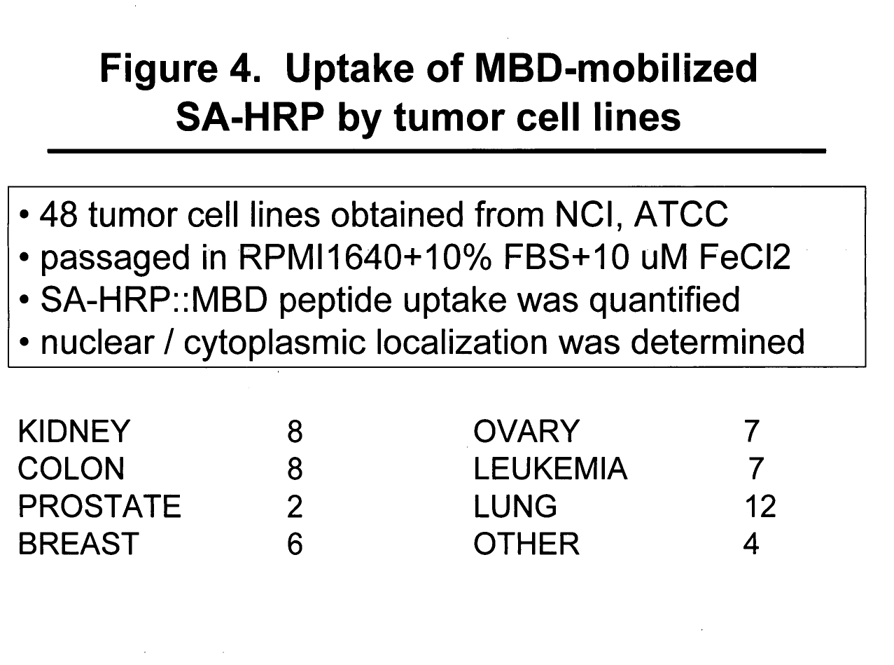

FIG. 5 shows cell internalization of MBD-mobilized SA-HRP in tumor cell lines. For each of the selected anatomical sites, a pair of cell lines was chosen based on the results shown in Table 2.

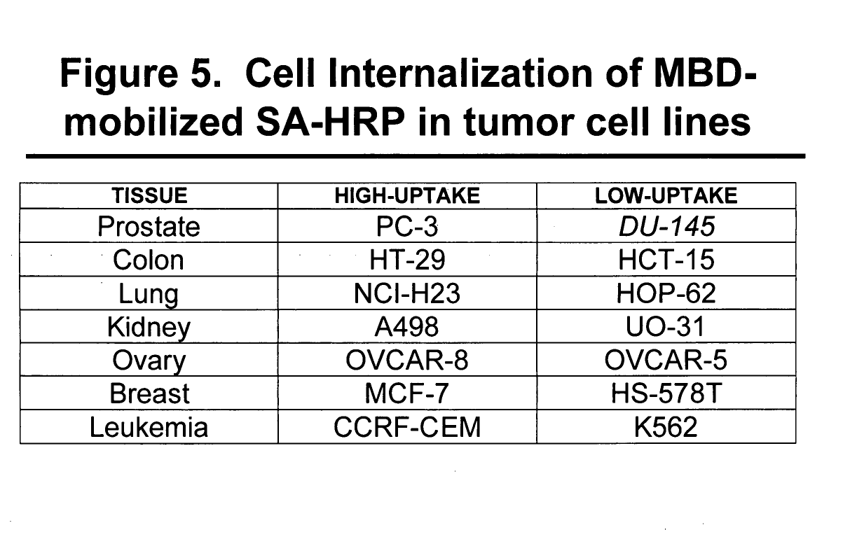

FIG. 6 shows cell internalization of MBD-mobilized SA-HRP in tumor cell lines. Using pairwise comparison of gene array results from 7 pairs of cell lines (each pair from a different anatomical site, as shown in Table 3), the functional distribution of differentially regulated genes is shown.

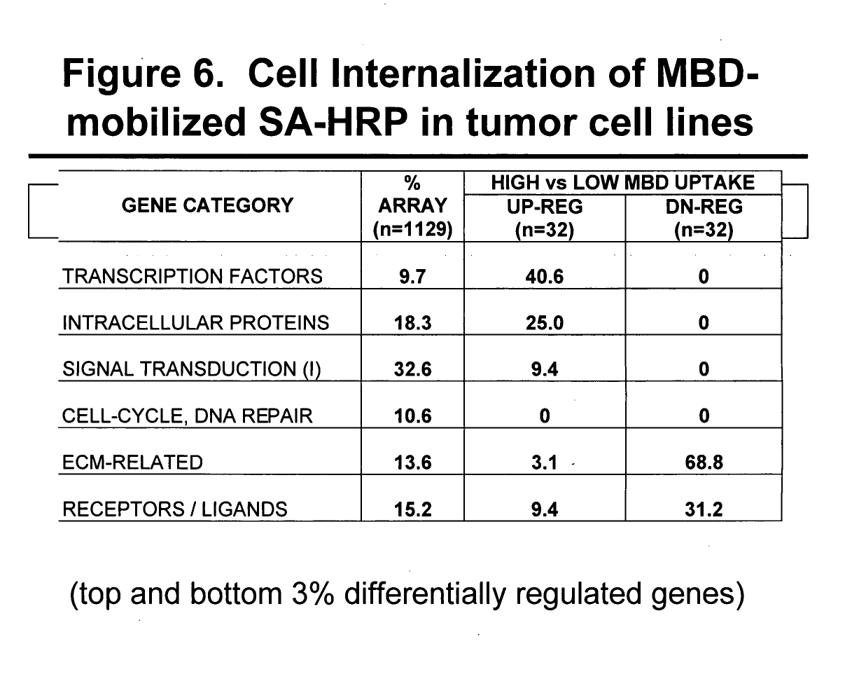

FIG. 7 shows up-regulated genes correlated to MBD-mobilized HRP internalization in tumor cell lines. The vast majority of up-regulated genes associated with greater uptake are associated with cellular stress responses.

FIG. 8 shows down-regulated genes correlated to MBD-mobilized HRP internalization in tumor cell lines. The vast majority of down-regulated genes are associated with secreted gene products.

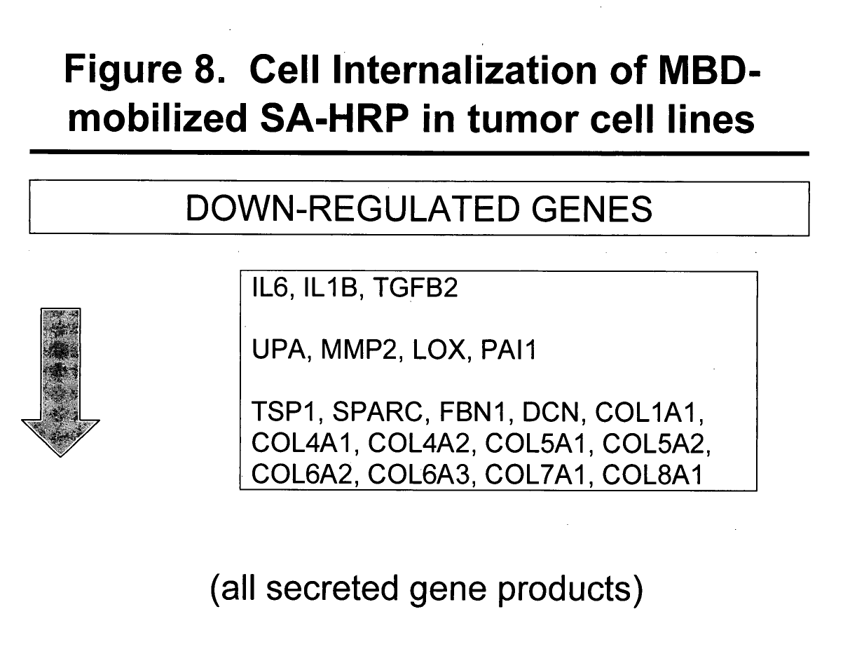

FIG. 9 shows examples of specific genes that are up- or down-regulated in association with cell internalization of MBD-mobilized SA-HRP in tumor cell lines.

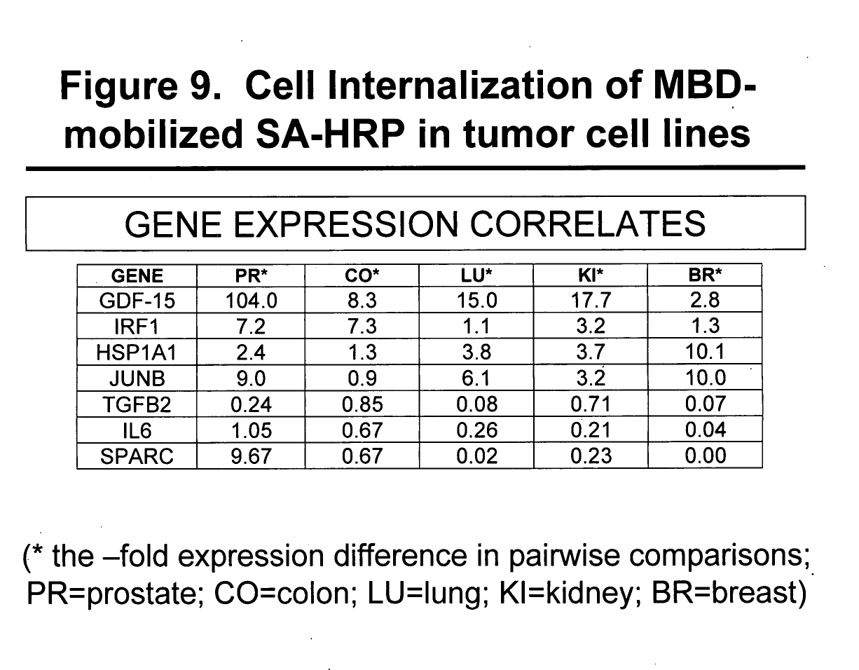

FIG. 10 shows surface markers cross-linked in association with cell internalization of MBD-mobilized SA-HRP in tumor cell lines. Membrane Markers: Cross-linking to biotinylated MBD21 peptide was performed on chilled cells as previously described (Singh B. et al op. cit.). Cell extracts were captured on Ni-NTA-coated 96-well plates, washed, blocked with 3% BSA and probed with the relevant antibody to the surface markers indicated. Intracellular Markers: Extracts were measured using standard ELISAs.

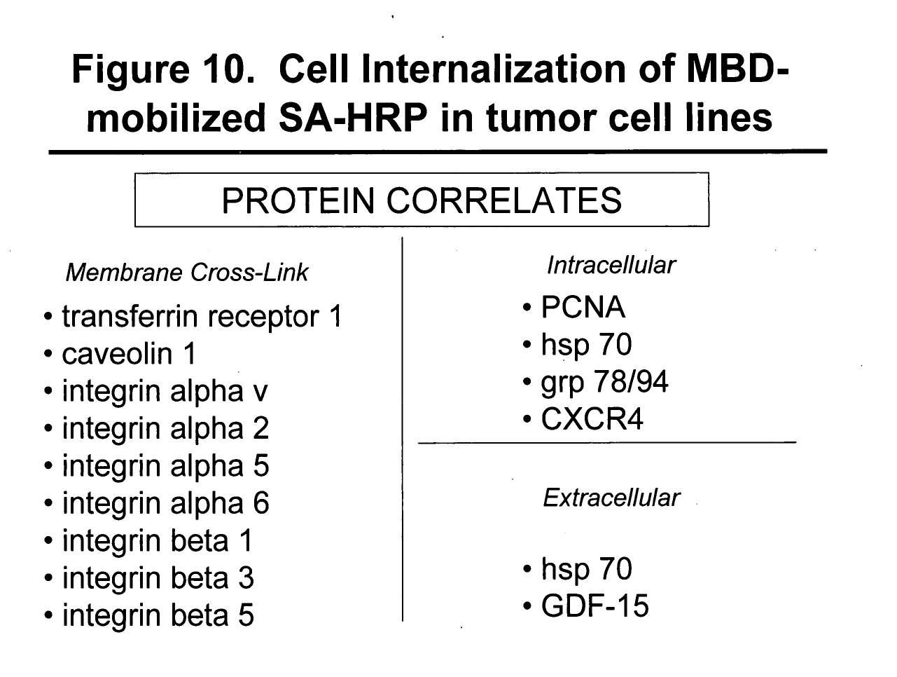

FIG. 11 shows average GDF-15/MIC-1/PLAB secretion by the high- and low-uptake cell lines of Table 3. There is a statistically significant difference between the high- and low-uptake cell line cohorts.

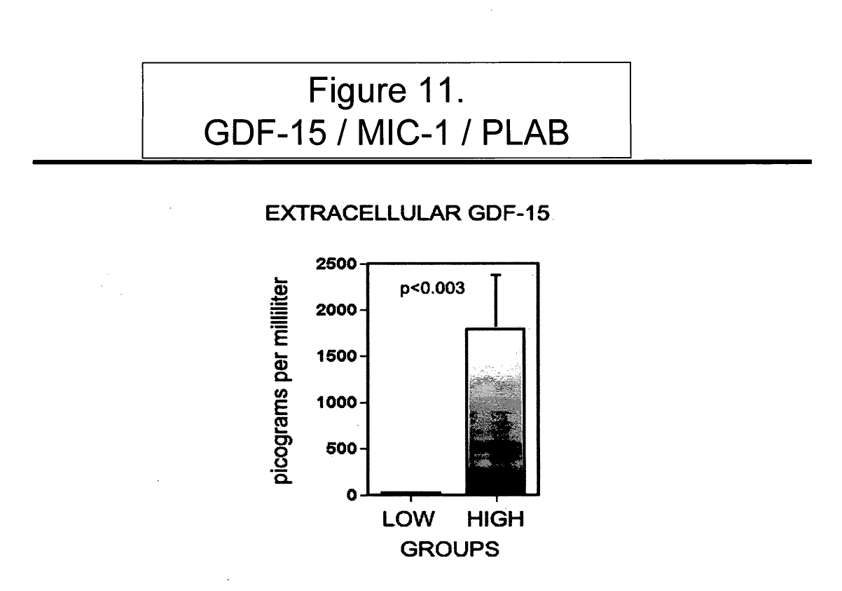

FIG. 12 shows GDF-15/MIC-1/PLAB levels are correlated (r=0.87) to MBD-mediated uptake in the same collection of cell lines reported in FIG. 11. Together with the results shown in FIG. 11, these results point to a potential usefulness of GDF15 as a diagnostic marker.

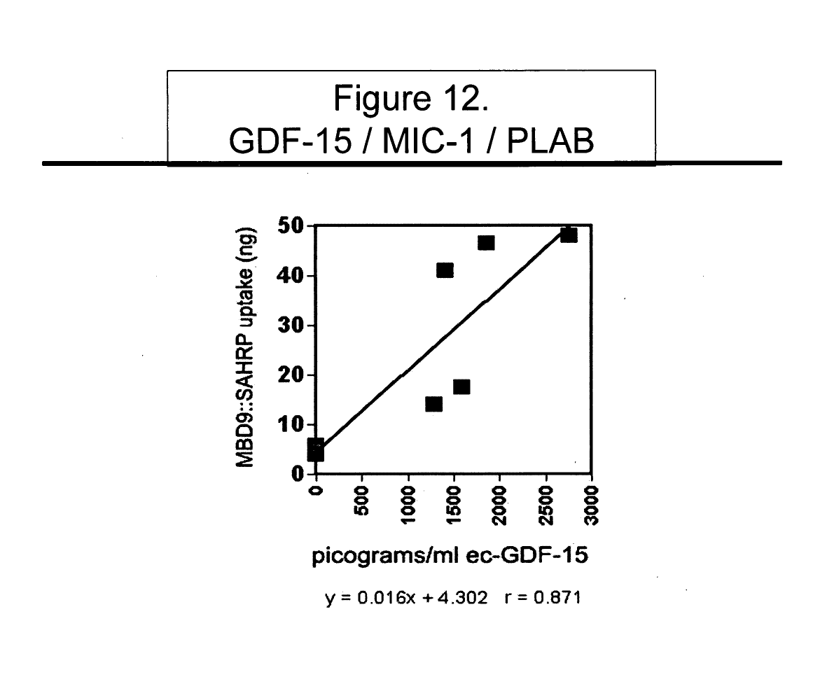

FIG. 13 shows some candidates cellular stress response programs.

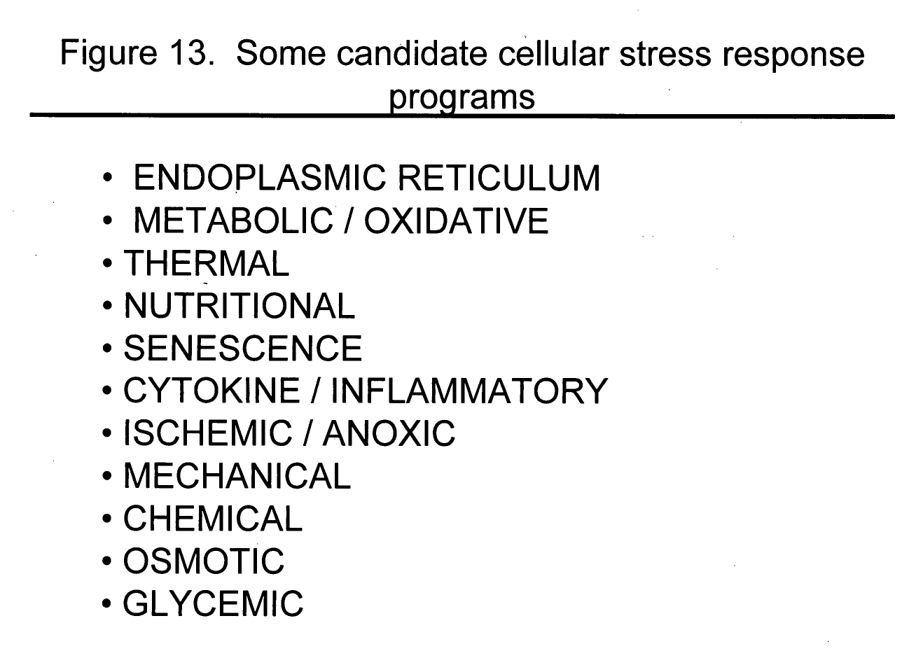

FIG. 14 shows cell internalization of MBD-mobilized SA-HRP in five tumor cell lines and the effect of heatshock pre-treatment.

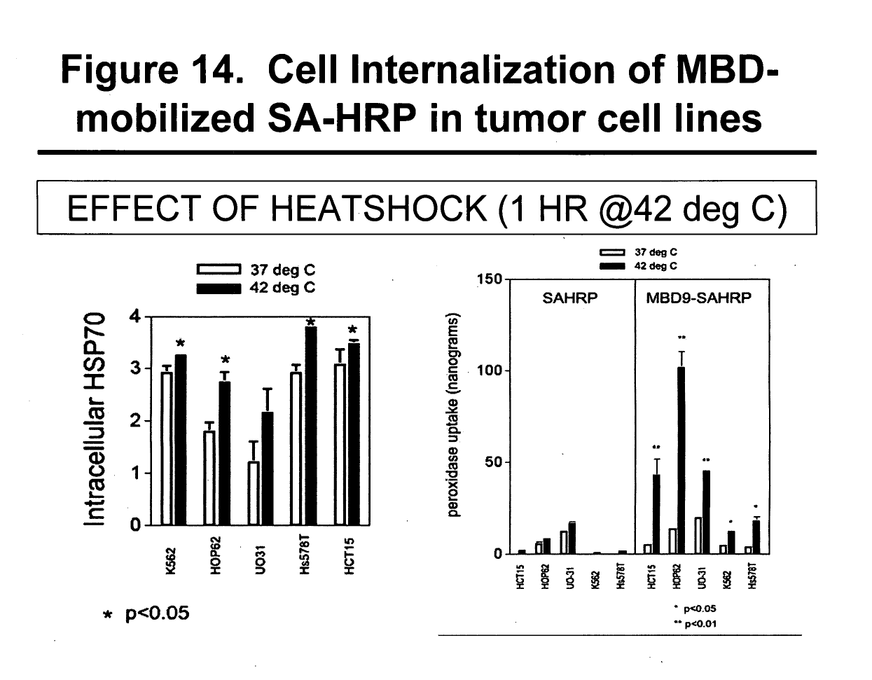

FIG. 15 shows cell internalization of MBD-mobilized SA-HRP in UO-31 cell line after thapsigargin pretreatment for the indicated times (endoplasmic reticulum (ER) stress). Cellular fractionation of extracts from each time point reveal differences in partitioning at different times between nuclear and non-nuclear intracellular location of the MBD-mobilized proteins.

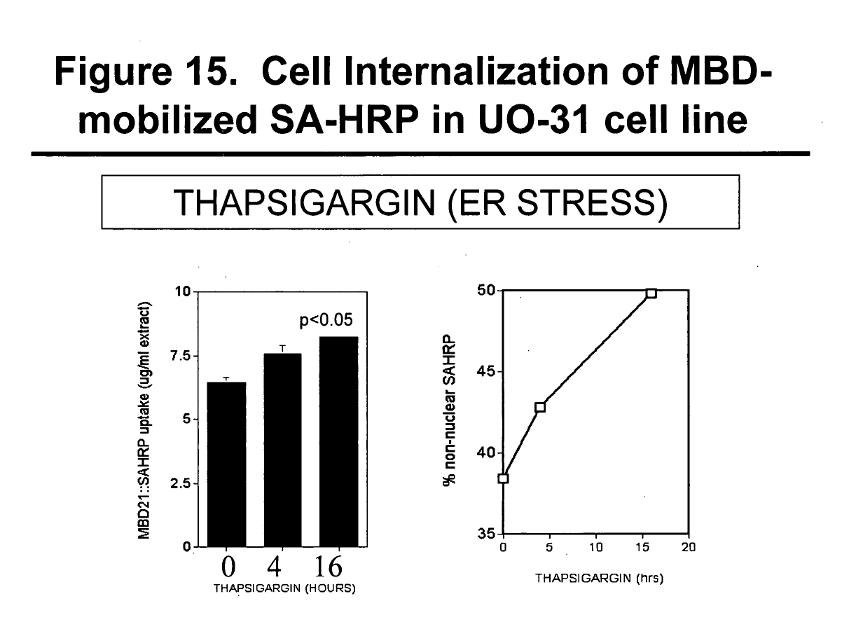

FIG. 16 shows biodistribution of MBD-tagged proteins systemically administered to rats in vivo. Male Lewis rats were sacrificed 2 hours after intravenous injection of the indicated tracer proteins at 1 mg/kg bolus. Tissues were analyzed for TK protein by ELISA.

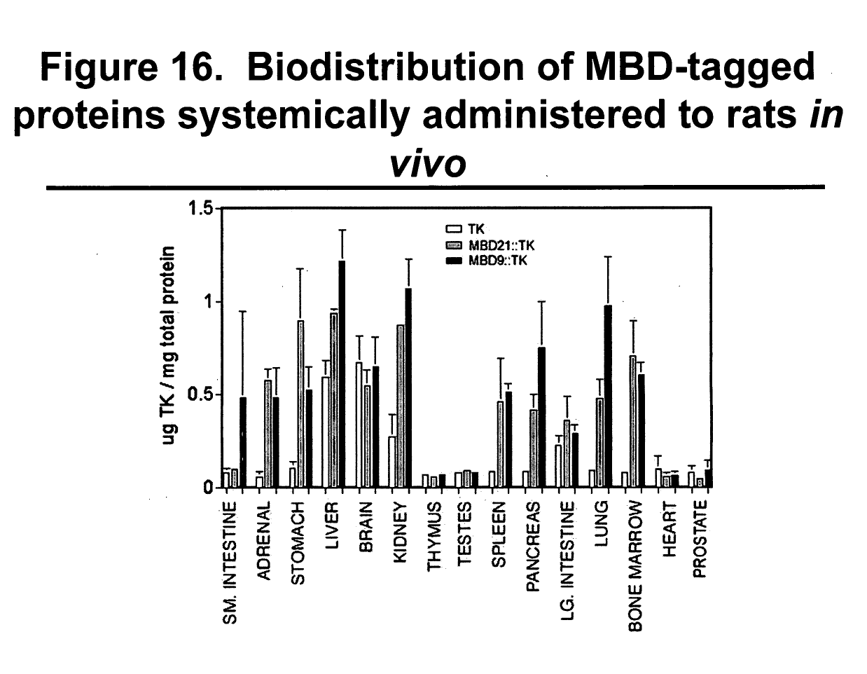

FIG. 17 shows blood cell association of MBD-tagged proteins systemically administered in vivo in the same experiment described in FIG. 16. A strong MBD-specific association with red blood cells is observed.

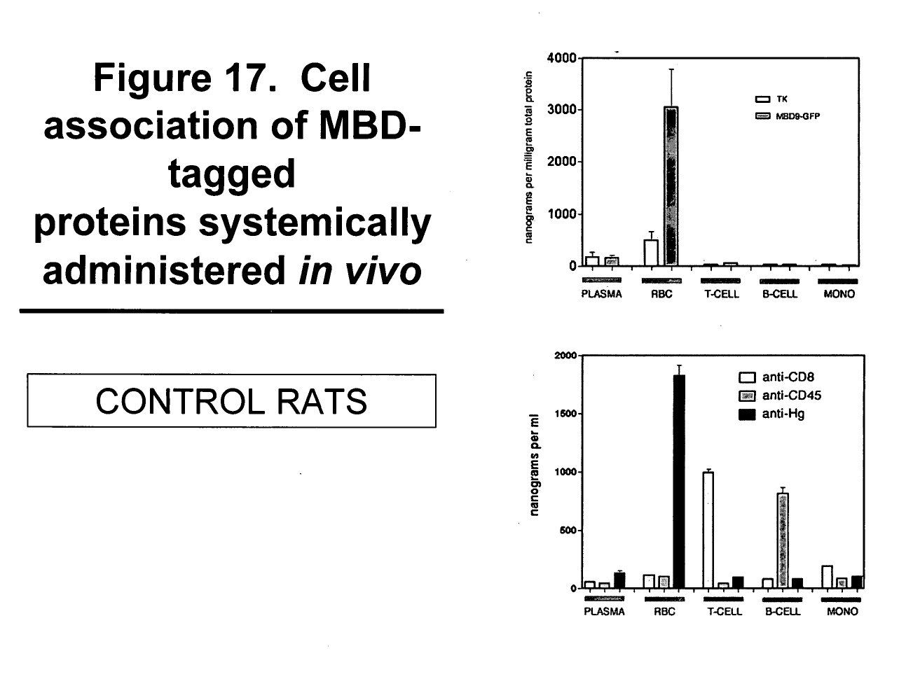

FIG. 18 shows markers of disease progression in a rat adjuvant arthritis model.

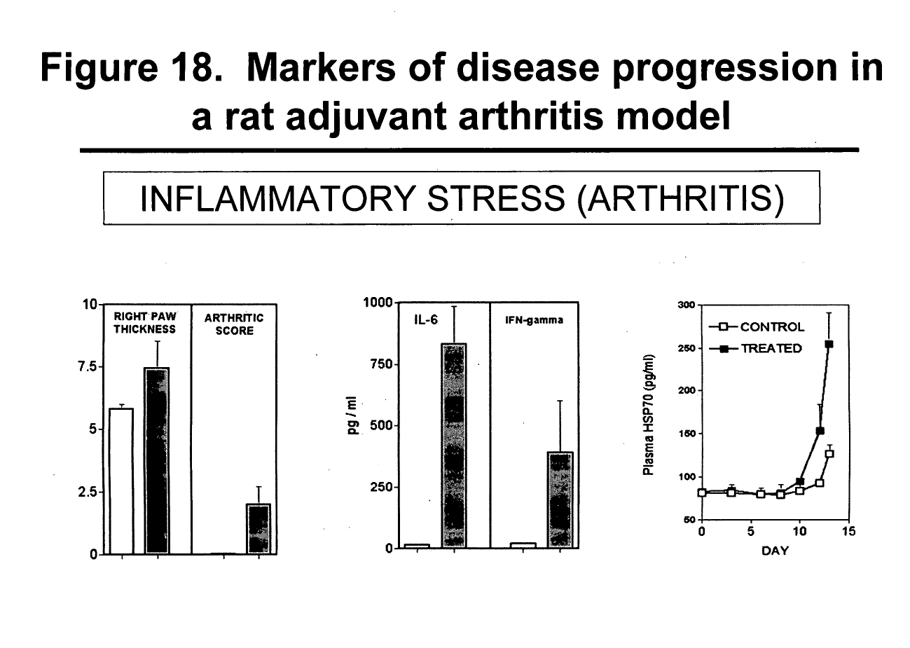

FIG. 19 shows cell internalization of MBD-tagged GFP protein systemically administered in vivo as described in FIG. 16, but using the rat adjuvant arthritis model of FIG. 18. The effects of inflammatory stress (arthritis) on organ-specific uptake of MBD-mobilized GFP protein can be measured in this experiment.

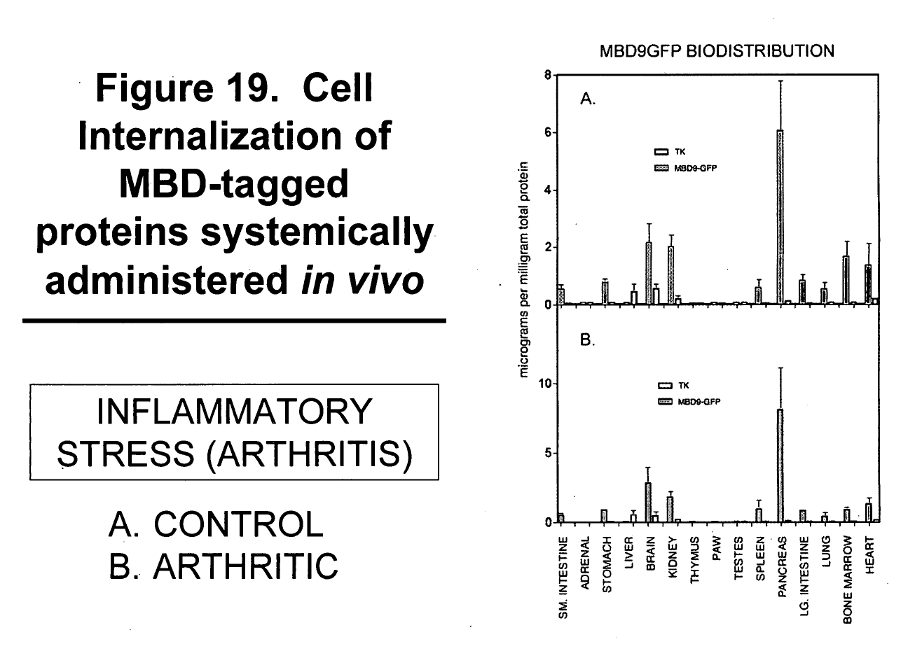

FIG. 20 shows cell internalization of MBD-tagged SA::HRP protein systemically administered in vivo in the same inflammatory stress (arthritis) model of FIG. 19.

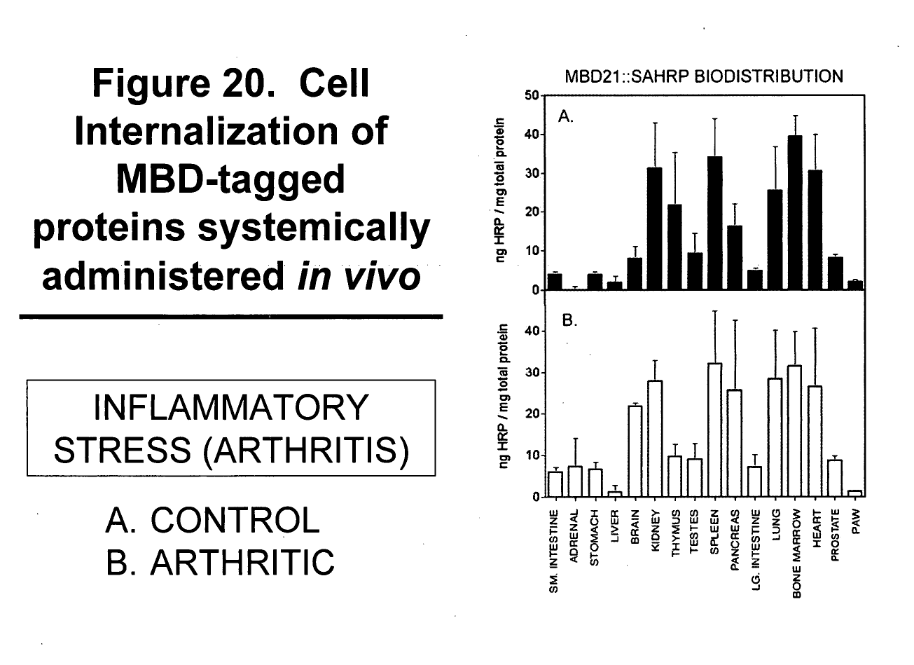

FIG. 21 shows stress-related cell internalization of MBD-tagged HRP protein by HEK293 cells.

FIG. 22 shows stress-related cell internalization of MBD-tagged HRP protein by PC-12 cells.

### MODES FOR CARRYING OUT THE INVENTION

Methods of Identifying Candidates for Treatment

The invention provides methods for identifying candidates for treatment with MBD peptide-based therapies.

Candidates for treatment with MBD peptide-based therapies are individuals (a) for whom MBD peptide-based therapy has been proposed (such as individuals who have been diagnosed with a disorder treatable with an MBD peptide-based therapy) and whose relevant tissue is predicted to have relatively high uptake of MBD peptide(s).

MBD peptide based therapy has been previously disclosed for a number of different indications, including cancer (such as breast, prostate, colon, ovarian, pancreatic, gastric and lung cancer), autoimmune disease, cardiovascular indications, arthritis, asthma, allergy, reproductive indications, retinal proliferative disease, bone disease, inflammatory disease, inflammatory bowel disease, and fibrotic disease. MBD peptides and therapies based thereon are further describe in U.S. patent application publication nos. 2003/0059430, 2003/0161829, and 2003/0224990.

The inventor has discovered a number of different genes which are differentially regulated between cells that have low uptake of MBD peptides and those that have high uptake of MBD peptides. These genes, referred to herein as “MBD uptake indicator genes”, include GDF15, SRC, ATF3, HSPF3, FAPP2, PSMB9, PSMB10, c-JUN, JUN-B, HSPA1A, HSPA6, NFKB2, IRF1, WDR9A, MAZ, NSG-X, KIAA1856, BRF2, COL9A3, TPD52, TAX40, PTPN3, CREM, HCA58, TCFL5, CEBPB, IL6R, ABCP2, CTGF, LAMA4, LAMB3, IL6, IL1B, UPA, MMP2, LOX, SPARC, FBN1, LUM, PAI1, TGFB2, URB, TSP1, CSPG2, DCN, ITGA5, TKT, CAV1, CAV2, COL1A1, COL4A1, COL4A2, COL5A1, COL5A2, COL6A2, COL6A3, COL7A1, COL8A1, and IL7R. Of these genes, GDF15, SRC, ATF3, HSPF3, FAPP2, PSMB9, PSMB10, c-JUN, JUN-B, HSPA1A, HSPA6, NFKB2, IRF1, WDR9A, MAZ, NSG-X, KIAA1856, BRF2, COL9A3, TPD52, TAX40, PTPN3, CREM, HCA58, TCFL5, CEBPB, IL6R and ABCP2 are up-regulated in cells which have high uptake of MBD peptides. It should be noted that at least one third of these up-regulated genes have been previously associated with cellular responses to stress (e.g. GDF15, ATF3, HSPF3, PSMB9, PSMB10, c-JUN, JUN-B, HSPA1A, HSPA6, NFKB2, IRF1). Down-regulated genes include CTGF, LAMA4, LAMB3, IL6, IL1B, UPA, MMP2, LOX, SPARC, FBN1, LUM, PAI1, TGFB2, URB, TSP1, CSPG2, DCN, ITGA5, TKT, CAV1, CAV2, COL1A1, COL4A1, COL4A2, COL5A1, COL5A2, COL6A2, COL6A3, COL7A1, COL8A1, and IL7R. The inventor further notes that specific formulae for identifying candidates for MBD peptide therapy may be developed using the data and techniques described herein.

Accordingly, the invention provides methods of identifying candidates for MBD peptide-based therapy by obtaining a measured level for at least one MBD uptake indicator gene in a tissue sample from an individual and comparing that measured level with a reference level. For up-regulated genes, a comparison that indicates that the measured level is higher than the reference level identifies a candidate for MBD peptide-based therapy. Likewise, a comparison that indicates that the measured level is lower than a reference level for a down-regulated MBD uptake indicator gene is lower than the reference level identifies a candidate for MBD peptide-based therapy.

Levels of the particular genes which are differentially regulated may be measured using any technology known in the art. Generally, mRNA is extracted from a sample of the relevant tissue (e.g., where the individual has been diagnosed with cancer, a biopsy sample of the tumor will generally be the sample tested). Direct quantitation methods (methods which measure the level of transcripts from a particular gene without conversion of the RNA into DNA or any amplification) may be used, but it is believed that measurement will be more commonly performed using technology which utilizes an amplification step (thereby reducing the minimum size sample necessary for testing).

Amplification methods generally involve a preliminary step of conversion of the mRNA into cDNA by extension of a primer (commonly one including an oligo-dT portion) hybridized to the mRNA in the sample with a RNA-dependent DNA polymerase. Additionally, a second cDNA strand (complementary to the first synthesized strand) may be synthesized when desired or necessary. Second strand cDNA is normally synthesized by extension of a primer hybridized to the first cDNA strand using a DNA-dependent DNA polymerase. The primer for second strand synthesis may be a primer that is added to the reaction (such as random hexamers) or may be ‘endogenous’ to the reaction (i.e., provided by the original RNA template, such as by cleavage with an enzyme or agent that cleaves RNA in a RNA/DNA hybrid, such as RNase H).

Amplification may be carried out separately from quantitation (e.g., amplification by single primer isothermal amplification, followed by quantitation of the amplification product by probe hybridization), or may be part of the quantitation process, such as in real time PCR.

Measured levels may be obtained by the practitioner of the instant invention, or may be obtained by a third party (e.g., a clinical testing laboratory) who supplies the measured value(s) to the practitioner.

Reference levels are generally obtained from “normal” tissues. Normal tissues are those which are not afflicted with the particular disease or disorder which is the subject of the MBD peptide-based therapy. For example, when the disease to be treated with MBD peptide-based therapy is ductal breast carcinoma, the reference value is normally obtained from normal breast duct tissue. Likewise, for cardiovascular disorders, the “normal” tissue might be normal arterial wall tissue (e.g., when the disorder is atherosclerosis). Alternately, values from cells (which may be tissue culture cells or cell lines) which have low MBD peptide uptake may also be used to derive a reference value.

The process of comparing a measured value and a reference value can be carried out in any convenient manner appropriate to the type of measured value and reference value for the MBD uptake indicator gene at issue. It should be noted that the measured values obtained for the MBD uptake indicator gene(s) can be quantitative or qualitative measurement techniques, thus the mode of comparing a measured value and a reference value can vary depending on the measurement technology employed. For example, when a qualitative colorimetric assay is used to measure MBD uptake indicator gene levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device). Quantitative values (e.g., transcripts/cell or transcripts/unit of RNA, or even arbitrary units) may also be used. As with qualitative measurements, the comparison can be made by inspecting the numerical data, by inspecting representations of the data (e.g., inspecting graphical representations such as bar or line graphs).

As will be understood by those of skill in the art, the mode of detection of the signal will depend on the exact detection system utilized in the assay. For example, if a radiolabeled detection reagent is utilized, the signal will be measured using a technology capable of quantitating the signal from the biological sample or of comparing the signal from the biological sample with the signal from a reference sample, such as scintillation counting, autoradiography (typically combined with scanning densitometry), and the like. If a chemiluminescent detection system is used, then the signal will typically be detected using a luminometer. Methods for detecting signal from detection systems are well known in the art and need not be further described here.

When more than one MBD uptake indicator gene is measured (i.e., measured values for two or more MBD uptake indicator genes are obtained), the sample may be divided into a number of aliquots, with separate aliquots used to measure different MBD uptake indicator gene (although division of the biological sample into multiple aliquots to allow multiple determinations of the levels of the MBD uptake indicator gene(s) in a particular sample are also contemplated). Alternately the sample (or an aliquot therefrom) may be tested to determine the levels of multiple MBD uptake indicator genes in a single reaction using an assay capable of measuring the individual levels of different MBD uptake indicator genes in a single assay, such as an array-type assay or assay utilizing multiplexed detection technology (e.g., an assay utilizing detection reagents labeled with different fluorescent dye markers).

As will be understood by those in the art, the exact identity of a reference value will depend on the tissue that is the target of treatment and the particular measuring technology used. In some embodiments, the comparison determines whether the measured value for the MBD uptake indicator gene is above or below the reference value. In some embodiments, the comparison is performed by finding the “fold difference” between the reference value and the measured value (i.e., dividing the measured value by the reference value). Table 1 lists certain exemplary fold differences for use in the instant invention.

TABLE 1

| GENE | Prostate | Colon | Lung | Kidney | Breast |
|---|---|---|---|---|---|
| GDF-15 | 50 | 4 | 7 | 8 | 1.4 |
| IRF1 | 3 | 3 | 1.05 | 1.6 | 1.15 |
| HSP1A1 | 1.7 | 1.15 | 2.4 | 2.8 | 5 |
| JUNB | 5 | 0.95 | 3 | 1.6 | 5 |
| TGFB2 | 0.6 | 0.92 | 0.5 | 0.85 | 0.5 |
| IL6 | 1.05 | 0.85 | 0.6 | 0.6 | 0.5 |
| SPARC | 5 | 0.85 | 0.5 | 0.6 | |

Candidates suitable for treatment with MBD peptide-based therapies are identified when at least a simple majority of the comparisons between the measured values and the reference values indicate that the cells in the sample (and thus the diseased cells in the individual) have relatively high uptake of MBD peptides. For up-regulated MBD uptake indicator genes (GDF15, SRC, ATF3, HSPF3, FAPP2, PSMB9, PSMB10, c-JUN, JUN-B, HSPA1A, HSPA6, NFKB2, IRF1, WDR9A, MAZ, NSG-X, KIAA1856, BRF2, COL9A3, TPD52, TAX40, PTPN3, CREM, HCA58, TCFL5, CEBPB, IL6R and ABCP2), a measured value that is greater than the reference value (which may be a simple “above or below” comparison or a comparison to find a minimum fold difference) indicates that the cells in the sample have relatively high uptake of MBD peptides. For down-regulated MBD uptake indicator genes (CTGF, LAMA4, LAMB3, IL6, IL1B, UPA, MMP2, LOX, SPARC, FBN1, LUM, PAI1, TGFB2, URB, TSP1, CSPG2, DCN, ITGA5, TKT, CAV1, CAV2, COL1A1, COL4A1, COL4A2, COL5A1, COL5A2, COL6A2, COL6A3, COL7A1, COL8A1, and IL7R), a measured value that is less than the reference value (which may be a simple “above or below” comparison or a comparison to find a minimum fold difference) indicates that the cells in the sample have relatively high uptake of MBD peptides.

Additionally, because certain of the MBD uptake indicator genes are found in serum (e.g. HSP70, GFP15), the invention also provides methods of identifying candidates for MBD peptide-based therapy by obtaining a measured level for at least one MBD uptake indicator gene in a biological fluid sample from an individual and comparing that measured level with a reference level. For up-regulated genes, a comparison that indicates that the measured level is higher than the reference level identifies a candidate for MBD peptide-based therapy. Likewise, a comparison that indicates that the measured level is lower than a reference level for a down-regulated MBD uptake indicator gene is lower than the reference level identifies a candidate for MBD peptide-based therapy.

A measured level is obtained for the relevant tissue for at least one MBD uptake indicator protein (i.e., the protein encoded by an MBD uptake marker gene), although multiple MBP uptake indicator proteins may be measured in the practice of the invention. Generally, it is preferred that measured levels are obtained for more than one MBD uptake indicator protein. Accordingly, the invention may be practiced using at least one, at least two, at least three, at least four, at least five, at least six, at least seven, at least eight, at least nine, at least ten, or more than ten MBD uptake indicator proteins. In certain embodiments, at least one of the measured values is obtained for a MBD uptake indicator protein that is up-regulated in cells which have high MBD peptide uptake levels and at least one of the measured values is obtained for a MBD uptake indicator protein that is down-regulated in cells which have high MBD peptide uptake levels. As will be apparent to those of skill in the art, the MBD uptake indicator proteins for which measured values are obtained are most commonly MBD uptake indicator proteins which may be secreted (e.g., HSP70, GDF15).

The MBD uptake indicator protein(s) may be measured using any available measurement technology that is capable of specifically determining the level of the MBD uptake indicator protein in a biological sample. In certain embodiments, the measurement may be either quantitative or qualitative, so long as the measurement is capable of indicating whether the level of the MBD uptake indicator protein in the biological sample is above or below the reference value.

Although some assay formats will allow testing of biological samples without prior processing of the sample, it is expected that most biological samples will be processed prior to testing. Processing generally takes the form of elimination of cells (nucleated and non-nucleated), such as erythrocytes, leukocytes, and platelets in blood samples, and may also include the elimination of certain proteins, such as certain clotting cascade proteins from blood.

Commonly, MBD uptake indicator protein levels will be measured using an affinity-based measurement technology. Affinity-based measurement technology utilizes a molecule that specifically binds to the MBD uptake indicator protein being measured (an “affinity reagent,” such as an antibody or aptamer), although other technologies, such as spectroscopy-based technologies (e.g., matrix-assisted laser desorption ionization-time of flight, or MALDI-TOF, spectroscopy) or assays measuring bioactivity (e.g., assays measuring mitogenicity of growth factors) may be used.

Affinity-based technologies include antibody-based assays (immunoassays) and assays utilizing aptamers (nucleic acid molecules which specifically bind to other molecules), such as ELONA. Additionally, assays utilizing both antibodies and aptamers are also contemplated (e.g., a sandwich format assay utilizing an antibody for capture and an aptamer for detection).

If immunoassay technology is employed, any immunoassay technology which can quantitatively or qualitatively measure the level of a MBD uptake indicator protein in a biological sample may be used. Suitable immunoassay technology includes radioimmunoassay, immunofluorescent assay, enzyme immunoassay, chemiluminescent assay, ELISA, immuno-PCR, and western blot assay.

Likewise, aptamer-based assays which can quantitatively or qualitatively measure the level of a MBD uptake indicator protein in a biological sample may be used in the methods of the invention. Generally, aptamers may be substituted for antibodies in nearly all formats of immunoassay, although aptamers allow additional assay formats (such as amplification of bound aptamers using nucleic acid amplification technology such as PCR (U.S. Pat. No. 4,683,202) or isothermal amplification with composite primers (U.S. Pat. Nos. 6,251,639 and 6,692,918).

A wide variety of affinity-based assays are known in the art. Affinity-based assays will utilize at least one epitope derived from the MBD uptake indicator protein of interest, and many affinity-based assay formats utilize more than one epitope (e.g., two or more epitopes are involved in “sandwich” format assays; at least one epitope is used to capture the marker, and at least one different epitope is used to detect the marker).

Affinity-based assays may be in competition or direct reaction formats, utilize sandwich-type formats, and may further be heterogeneous (e.g., utilize solid supports) or homogenous (e.g., take place in a single phase) and/or utilize or immunoprecipitation. Most assays involve the use of labeled affinity reagent (e.g., antibody, polypeptide, or aptamer); the labels may be, for example, enzymatic, fluorescent, chemiluminescent, radioactive, or dye molecules. Assays which amplify the signals from the probe are also known; examples of which are assays which utilize biotin and avidin, and enzyme-labeled and mediated immunoassays, such as ELISA and ELONA assays.

In a heterogeneous format, the assay utilizes two phases (typically aqueous liquid and solid). Typically a MBD uptake indicator protein-specific affinity reagent is bound to a solid support to facilitate separation of the MBD uptake indicator protein from the bulk of the biological sample. After reaction for a time sufficient to allow for formation of affinity reagent/MBD uptake indicator protein complexes, the solid support containing the antibody is typically washed prior to detection of bound polypeptides. The affinity reagent in the assay for measurement of MBD uptake indicator proteins may be provided on a support (e.g., solid or semi-solid); alternatively, the polypeptides in the sample can be immobilized on a support. Examples of supports that can be used are nitrocellulose (e.g., in membrane or microtiter well form), polyvinyl chloride (e.g., in sheets or microtiter wells), polystyrene latex (e.g., in beads or microtiter plates), polyvinylidine fluoride, diazotized paper, nylon membranes, activated beads, and Protein A beads. Both standard and competitive formats for these assays are known in the art.

Array-type heterogeneous assays are suitable for measuring levels of MBD uptake indicator proteins when the methods of the invention are practiced utilizing multiple MBD uptake indicator proteins. Array-type assays used in the practice of the methods of the invention will commonly utilize a solid substrate with two or more capture reagents specific for different MBD uptake indicator proteins bound to the substrate a predetermined pattern (e.g., a grid). The biological sample is applied to the substrate and MBD uptake indicator proteins in the sample are bound by the capture reagents. After removal of the sample (and appropriate washing), the bound MBD uptake indicator proteins are detected using a mixture of appropriate detection reagents that specifically bind the various MBD uptake indicator proteins. Binding of the detection reagent is commonly accomplished using a visual system, such as a fluorescent dye-based system. Because the capture reagents are arranged on the substrate in a predetermined pattern, array-type assays provide the advantage of detection of multiple MBD uptake indicator proteins without the need for a multiplexed detection system.

In a homogeneous format the assay takes place in single phase (e.g., aqueous liquid phase). Typically, the biological sample is incubated with an affinity reagent specific for the MBD uptake indicator protein in solution. For example, it may be under conditions that will precipitate any affinity reagent/antibody complexes which are formed. Both standard and competitive formats for these assays are known in the art.

In a standard (direct reaction) format, the level of MBD uptake indicator protein/affinity reagent complex is directly monitored. This may be accomplished by, for example, determining the amount of a labeled detection reagent that forms is bound to MBD uptake indicator protein/affinity reagent complexes. In a competitive format, the amount of MBD uptake indicator protein in the sample is deduced by monitoring the competitive effect on the binding of a known amount of labeled MBD uptake indicator protein (or other competing ligand) in the complex. Amounts of binding or complex formation can be determined either qualitatively or quantitatively.

Complexes formed comprising MBD uptake indicator protein and an affinity reagent are detected by any of a number of known techniques known in the art, depending on the format of the assay and the preference of the user. For example, unlabelled affinity reagents may be detected with DNA amplification technology (e.g., for aptamers and DNA-labeled antibodies) or labeled “secondary” antibodies which bind the affinity reagent. Alternately, the affinity reagent may be labeled, and the amount of complex may be determined directly (as for dye-(fluorescent or visible), bead-, or enzyme-labeled affinity reagent) or indirectly (as for affinity reagents “tagged” with biotin, expression tags, and the like).

As will be understood by those of skill in the art, the mode of detection of the signal will depend on the exact detection system utilized in the assay. For example, if a radiolabeled detection reagent is utilized, the signal will be measured using a technology capable of quantitating the signal from the biological sample or of comparing the signal from the biological sample with the signal from a reference sample, such as scintillation counting, autoradiography (typically combined with scanning densitometry), and the like. If a chemiluminescent detection system is used, then the signal will typically be detected using a luminometer. Methods for detecting signal from detection systems are well known in the art and need not be further described here.

When more than one MBD uptake indicator protein is measured, the biological sample may be divided into a number of aliquots, with separate aliquots used to measure different MBD uptake indicator proteins (although division of the biological sample into multiple aliquots to allow multiple determinations of the levels of the MBD uptake indicator protein in a particular sample are also contemplated). Alternately the biological sample (or an aliquot therefrom) may be tested to determine the levels of multiple MBD uptake indicator proteins in a single reaction using an assay capable of measuring the individual levels of different MBD uptake indicator proteins in a single assay, such as an array-type assay or assay utilizing multiplexed detection technology (e.g., an assay utilizing detection reagents labeled with different fluorescent dye markers).

It is common in the art to perform ‘replicate’ measurements when measuring MBD uptake indicator proteins. Replicate measurements are ordinarily obtained by splitting a sample into multiple aliquots, and separately measuring the MBD uptake indicator protein (s) in separate reactions of the same assay system. Replicate measurements are not necessary to the methods of the invention, but many embodiments of the invention will utilize replicate testing, particularly duplicate and triplicate testing.

Kits for Identification of Candidates for MBD Peptide Therapy

The invention provides kits for carrying out the methods of the invention. Kits of the invention comprise at least one probe specific for a MBD uptake indicator gene (and/or at least one affinity reagent specific for a MBD uptake indicator protein) and instructions for carrying out a method of the invention. More commonly, kits of the invention comprise at least two different MBD uptake indicator gene probes (or at least two affinity reagents specific for MBD uptake indicator proteins), where each probe/reagent is specific for a different MBD uptake indicator gene.

Kits comprising a single probe for a MBD uptake indicator gene (or affinity reagent specific for a MBD uptake indicator protein) will generally have the probe/reagent enclosed in a container (e.g., a vial, ampoule, or other suitable storage container), although kits including the probe/reagent bound to a substrate (e.g., an inner surface of an assay reaction vessel) are also contemplated. Likewise, kits including more than one probe/reagent may also have the probes/reagents in containers (separately or in a mixture) or may have the probes/affinity reagents bound to a substrate (e.g., such as an array or microarray).

A modified substrate or other system for capture of MBD uptake indicator gene transcripts or MBD uptake indicator proteins may also be included in the kits of the invention, particularly when the kit is designed for use in an array format assay.

In certain embodiments, kits according to the invention include the probes/reagents in the form of an array. The array includes at least two different probes/reagents specific for a MBD uptake indicator gene/protein (each probe/reagent specific for a different MBD uptake indicator gene/protein) bound to a substrate in a predetermined pattern (e.g., a grid). The localization of the different probes/reagents allows measurement of levels of a number of different MBD uptake indicator genes/.proteins in the same reaction.

The instructions relating to the use of the kit for carrying out the invention generally describe how the contents of the kit are used to carry out the methods of the invention. Instructions may include information as sample requirements (e.g., form, pre-assay processing, and size), steps necessary to measure the MBD uptake indicator gene(s), and interpretation of results.

Instructions supplied in the kits of the invention are typically written instructions on a label or package insert (e.g., a paper sheet included in the kit), but machine-readable instructions (e.g., instructions carried on a magnetic or optical storage disk) are also acceptable. In certain embodiments, machine-readable instructions comprise software for a programmable digital computer for comparing the measured values obtained using the reagents included in the kit.

Therapeutic Methods

The therapeutic methods of the invention utilize treatment of certain disorders (e.g., disorders characterized by secreted HSP70 and macrophage co-localized at the site of disease) with MBD peptide therapies. The invention provides methods of treating diseases characterized by measurable cellular stress responses (such as the induction of heat shock proteins) including, but not limited to, metabolic and oxidative stress, with MBD peptide therapies. MBD peptide therapies include treatment by administration of (a) MBD peptides, (b) MBD peptide fusions, and (c) MBD peptide conjugates.

The invention provides methods for delivering an MBD peptide-linked agent into live cells, said method comprising contacting said MBD peptide-linked agent to live cells that are under a condition of cellular stress, whereby said contact results in cellular uptake of said MBD-peptide-linked agent.

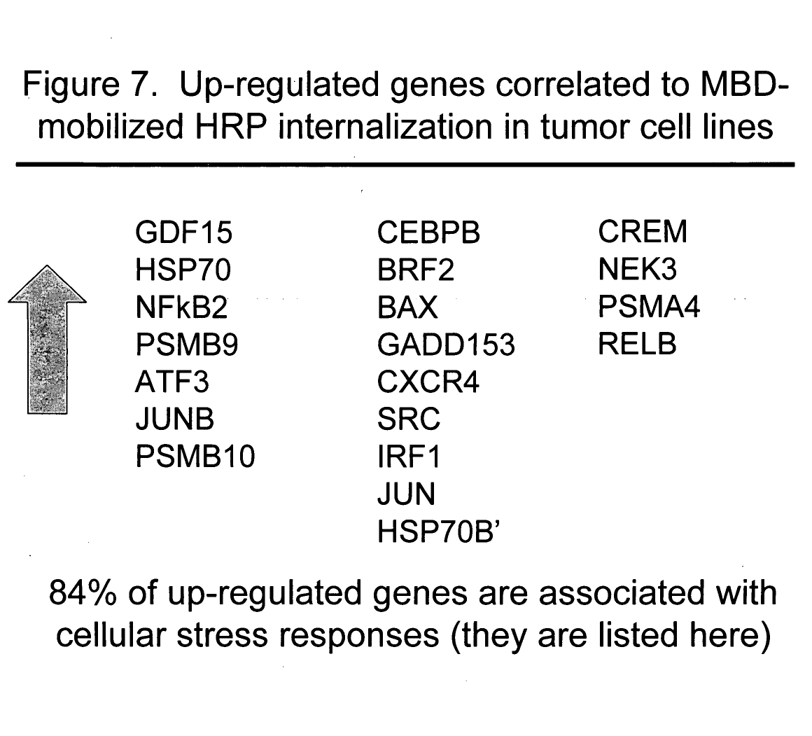

The condition of cellular stress can be any type of stress, such as thermal, immunological, cytokine, oxidative, metabolic, anoxic, endoplasmic reticulum, protein unfolding, nutritional, chemical, mechanical, osmotic and glycemic stress. In some embodiments, the condition of cellular stress is associated with upregulation of at least one, at least two, at least three, at least four, at least five, at least ten, at least fifteen, at least twenty, or all of the genes shown in FIG. 7 as compared to the cells not under the condition of cellular stress. Accordingly, the methods of invention may further include a step of comparing levels of gene expression of any one or more of the genes shown in FIG. 7 in cells under a condition of cellular stress to levels of gene expression of the same gene or genes in the cells not under the condition of cellular stress, whereby cells that are candidate targets for delivering MBD peptide-linked agents are identified. The upregulation may be at least about 1.5-fold, at least about 2-fold, at least about 3-fold, at least about 5-fold, or at least about 10-fold.

“Metal-binding domain peptide” or “MBD peptide” means an IGFBP-derived peptide or polypeptide from about 12 to about 60 amino acids long, preferably from about 13 to 40 amino acids long, comprising a segment of the CD-74-homology domain sequence in the carboxy-terminal 60-amino acids of IGFBP-3, comprising the sequence CRPSKGRKRGFC and exhibiting metal-binding properties, but differing from intact IGFBP-3 by exhibiting distinct antigenic properties, lacking IGF-I-binding properties, and lacking the mid-region sequences (amino acids 88-148 of IGFBP-3 sequence). For example, the peptide GFYKKKQCRPSKGRKRGFCW is an example of a metal-binding domain peptide. It binds metal ions but not IGF-I, and polyclonal antibodies raised to this peptide do not substantially cross-react with intact IGFBP-3, and vice versa. In certain embodiments, the MBD peptide includes a caveolin consensus binding sequence (#x#xxxx#, where ‘#’ is an aromatic amino acid) in addition to, or overlapping with, the MBD peptide sequence. The caveolin consensus sequence may be at the amino terminal or carboxy terminal end of the peptide. In certain preferred embodiments, the caveolin consensus binding sequence is at the carboxy terminal end of the peptide, and overlaps with the MBD core 14-mer sequence. Exemplary MBD peptides with caveolin consensus binding sequences include peptides comprising the sequence QCRPSKGRKRGFCWAVDKYG or KKGFYKKKQCRPSKGRKRGFCWAVDKYG.

MBD peptides may be modified, such as by making conservative substitutions for the natural amino acid residue at any position in the sequence, altering phosphorylation, acetylation, glycosylation or other chemical status found to occur at the corresponding sequence position of IGFBP-3 in the natural context, substituting D- for L-amino acids in the sequence, or modifying the chain backbone chemistry, such as protein-nucleic-acid (PNA).

“Conjugates” of an MBD peptide and a second molecule include both covalent and noncovalent conjugates between a MBD peptide and a second molecule (such as a transcriptional modulator or a therapeutic molecule). Noncovalent conjugates may be created by using a binding pair, such as biotin and avidin or streptavidin or an antibody (including Fab fragments, scFv, and other antibody fragments/modifications) and its cognate antigen.

Sequence “identity” and “homology”, as referred to herein, can be determined using BLAST (Altschul, et al., 1990, J. Mol. Biol. 215(3):403-410), particularly BLASTP 2 as implemented by the National Center for Biotechnology Information (NCBI), using default parameters (e.g., Matrix 0 BLOSUM62, gap open and extension penalties of 11 and 1, respectively, gap x_dropoff 50 and wordsize 3). Unless referred to as “consecutive” amino acids, a sequence optionally can contain a reasonable number of gaps or insertions that improve alignment.

An effective amount of the MBD therapy is administered to a subject having the disease. In some embodiments, the MBD therapy is administered at about 0.001 to about 40 milligrams per kilogram total body weight per day (mg/kg/day). In some embodiments the MBD therapy is administered at about 0.001 to about 40 mg/kg/day of MBD peptide (i.e., the MBD peptide portion of the therapy administered is about 0.001 to about 40 mg/kg/day).

The terms “subject” and “individual”, as used herein, refer to a vertebrate individual, including avian and mammalian individuals, and more particularly to sport animals (e.g., dogs, cats, and the like), agricultural animals (e.g., cows, horses, sheep, and the like), and primates (e.g., humans).

The term “treatment” is used herein as equivalent to the term “alleviating”, which, as used herein, refers to an improvement, lessening, stabilization, or diminution of a symptom of a disease. “Alleviating” also includes slowing or halting progression of a symptom.

The MBD peptide is normally produced by recombinant methods, which allow the production of all possible variants in peptide sequence. Techniques for the manipulation of recombinant DNA are well known in the art, as are techniques for recombinant production of proteins (see, for example, in Sambrook et al., MOLECULAR CLONING: A LABORATORY MANUAL, Vols. 1-3 (Cold Spring Harbor Laboratory Press, 2 ed., (1989); or F. Ausubel et al., CURRENT PROTOCOLS IN MOLECULAR BIOLOGY (Green Publishing and Wiley-Interscience: New York, 1987) and periodic updates). Derivative peptides or small molecules of known composition may also be produced by chemical synthesis using methods well known in the art.

Preferably, the MBD peptide is produced using a bacterial cell strain as the recombinant host cell. An expression construct (i.e., a DNA sequence comprising a sequence encoding the desired MBD peptide operably linked to the necessary DNA sequences for proper expression in the host cell, such as a promoter and/or enhancer elements at the 5′ end of the construct and terminator elements in the 3′ end of the construct) is introduced into the host cell. The DNA sequence encoding the MBD peptide may optionally linked to a sequence coding another protein (a “fusion partner”), to form a fusion protein. Preferably, the DNA sequence encoding the MBD peptide is linked to a sequence encoding a fusion partner as described in U.S. Pat. No. 5,914,254. The expression construct may be an extrachromosomal construct, such as a plasmid or cosmid, or it may be integrated into the chromosome of the host cell, for example as described in U.S. Pat. No. 5,861,273.

Accordingly, the invention provides methods of treatment with fusions and/or conjugates of MBD peptides with molecules (such as agents) which are desired to be internalized into cells. The fusion partner molecules may be polypeptides, nucleic acids, or small molecules which are not normally internalized (e.g., because of large size, hydrophilicity, etc.). As will be apparent to one of skill in the art, such fusions/conjugates will be useful in a number of different areas, including pharmaceuticals (to promote internalization of therapeutic molecules which do not normally become internalized), gene therapy (to promote internalization of gene therapy constructs), and research (allowing ‘marking’ of cells with an internalized marker protein). Preferred MBD peptides are peptides comprising the sequence KKGFYKKKQCRPSKGRKRGFCW or a sequence having at least 80, 85, 90, 95, 98, or 99% homology to said sequence. Fusions of MBD peptides and polypeptides are preferably made by creation of a DNA construct encoding the fusion protein, but such fusions may also be made by chemical ligation of the MBD peptide and the polypeptide of interest. Conjugates of MBD peptides and nucleic acids or small molecules can be made using chemical crosslinking technology known in the art. Preferably, the conjugate is produced using a heterobifunctional crosslinker to avoid production of multimers of the MBD peptide.

Therapy in accordance with the invention may utilize MBD peptides and transcriptional modulators (e.g., transcription factors). For example, T-bet (Szabo et al., 2000, Cell 100(6):655-69), a transcription factor that appears to commit T lymphocytes to the Tₕ₁ lineage, can be fused to a MBD peptide to create a molecule a useful therapeutic. Likewise, therapy in accordance with the invention using conjugates of MBD peptides and therapeutic molecules is also provided. MBD peptides may be conjugated with any therapeutic molecule which is desired to be delivered to the interior of a cell, including antisense oligonucleotides and polynucleotide constructs (e.g., encoding therapeutic molecules such as growth factors and the like).

Peptides comprising an MBD peptide which includes a caveolin consensus binding sequence (MBD/caveolin peptides) may also be incorporated into conjugates. MBD/caveolin peptides may be conjugated with any therapeutic molecule that is desired to be delivered to the interior of a cell, including antisense oligonucleotides and polynucleotide constructs (e.g., encoding therapeutic molecules such as growth factors and the like).

Molecules comprising an MBD peptide are preferably administered via oral or parenteral administration, including but not limited to intravenous (IV), intraperitoneal (IP), intramuscular (IM), subcutaneous (SC), intradermal (ID), transdermal, inhaled, and intranasal routes. IV, IP, IM, and ID administration may be by bolus or infusion administration. For SC administration, administration may be by bolus, infusion, or by implantable device, such as an implantable minipump (e.g., osmotic or mechanical minipump) or slow release implant. The MBD peptide may also be delivered in a slow release formulation adapted for IV, IP, IM, ID or SC administration. Inhaled MBD peptide is preferably delivered in discrete doses (e.g., via a metered dose inhaler adapted for protein delivery). Administration of a molecule comprising a MBD peptide via the transdermal route may be continuous or pulsatile. Administration of MBD peptides may also occur orally.

For parenteral administration, compositions comprising a MBD peptide may be in dry powder, semi-solid or liquid formulations. For parenteral administration by routes other than inhalation, the composition comprising a MBD peptide is preferably administered in a liquid formulation. Compositions comprising a MBD peptide formulation may contain additional components such as salts, buffers, bulking agents, osmolytes, antioxidants, detergents, surfactants, and other pharmaceutical excipients as are known in the art.

A composition comprising a MBD peptide is administered to subjects at a dose of about 0.001 to about 40 mg/kg/day, more preferably about 0.01 to about 10 mg/kg/day, more preferably 0.05 to about 4 mg/kg/day, even more preferably about 0.1 to about 1 mg/kg/day.

As will be understood by those of skill in the art, the symptoms of disease alleviated by the instant methods, as well as the methods used to measure the symptom(s) will vary, depending on the particular disease and the individual patient.

Patients treated in accordance with the methods of the instant invention may experience alleviation of any of the symptoms of their disease.

### EXAMPLES

### Example 1

HEK293 kidney cell line and 54 tumor cell lines obtained from the National Cancer Institute and passaged in RPMI1640 cell culture medium supplemented with 10% fetal bovine serum and 10 uM FeCl₂. Uptake of streptavidin-horseradish peroxidase (SA-HRP) conjugate and of various SA-HRP::MBD peptide complexes was determined as described (Singh et al. J Biol. Chem. 279 (1):477-87 [2004]) using biotinylated MBD9 (KKGFYKKKQCRPSKGRKRGFCWNGRK) and MBD21 (KKGFYKKKQCRPSKGRKRGFCWAVDKYG) peptides and SA-HRP. Nuclear and cytoplasmic localization of these proteins was also determined in each case. The results of this survey are summarized in Table 2. They show that the rate of MBD-mediated uptake is highly variable across cell lines. In order to establish the underlying molecular mechanism for this variability, we cross-linked MBD21 peptide to the following cell surface markers at 4 degrees Celsius as previously described (Singh et al. J Biol. Chem. 279 (1):477-87 [2004]): transferrin receptor 1, caveolin 1, PCNA, integrins alpha v, 2, 5 and 6, integrins beta 1, 3 and 5. Significant correlations (positive or negative) between crosslinking rates and the previously measured rates of MBD-mediated SA-HRP uptake were observed in the case of transferrin receptor 1, caveolin 1, integrins beta 3, beta 5 and alpha v. Based on the strength of these correlations, it was possible to derive crude predictive formulae for MBD-mediated uptake based on the rate of cross-linking to surface markers. Such predictive formulas could form the basis for a diagnostic procedure to select appropriate targets for MBD-based therapies.

TABLE 2

| | | MBD9 | MBD9 | MBD21 | MBD21 |
|---|---|---|---|---|---|
| | | Cyt. | Nuc. | Cyt. | Nuc. |
| Cell Line | Histologic Type | (ng) | (ng) | (ng) | (ng) |
| SK-OV-3 | hu Ascites Adenocarcinoma | 2.0 | 0.4 | <0.04 | <0.04 |
| OVCAR-3 | hu Ascites Adenocarcinoma | 2.4 | 4.6 | <0.04 | 3.2 |
| HOP 92 | hu Lung Large Cell, Undifferentiated | 2.5 | 1.6 | 1.5 | 1.6 |
| NCI-H226 | hu Lung Sqamous Cell | 2.6 | 1.8 | 0.7 | 0.9 |
| K562 | Lymph Leukemia | 2.6 | 1.3 | 2.8 | 1.1 |
| CCRF-SB | Lymph Leukemia | 2.6 | 0.6 | 1.7 | 0.1 |
| OVCAR-5 | hu Adenocarcinoma | 2.7 | 1.2 | 1.3 | 1.5 |
| 786-O | hu Renal Adenocarcinoma | 2.9 | 3.9 | 1.8 | 4.8 |
| COLO 205 | hu Ascitic Fluid Adenocarcinoma | 2.9 | 0.9 | 2.1 | 0.9 |
| DU-145 | hu Prostate Carcinoma | 3.1 | <0.04 | 25.7 | 3.3 |
| SW-620 | hu Colon Adenocarcinoma | 3.2 | 0.7 | 6.3 | 2.3 |
| WIDR | hu Colon Adenoarcinoma | 3.4 | 0.7 | 2.8 | 1.0 |
| HS 913T | hu Lung Mixed Cell | 3.4 | 1.1 | 2.1 | 1.8 |
| KM12 | hu Adenocarcinoma | 3.6 | 1.0 | 2.1 | 0.7 |
| OVCAR-8 | hu Adenocarcinoma | 3.9 | 5.0 | 6.1 | 13.1 |
| HCT-15 | hu Colon Adenocarcinoma | 4.0 | 0.8 | 2.7 | 0.7 |
| TK-10 | hu Renal Carcinoma | 4.0 | 1.3 | 5.0 | 2.2 |
| UO-31 | hu Renal Carcinoma | 4.6 | 1.0 | 1.3 | 3.3 |
| HCC 2998 | hu Adenocarcinoma | 4.6 | 3.7 | 2.1 | 2.4 |
| NHI-H322M | hu Lung Bronchi Alveolar Carcinoma | 5.2 | 5.0 | 6.0 | 8.3 |
| HT-29 | hu Recto-Sigmoid Colon Adenocarcinoma | 6.1 | 7.7 | 3.5 | 9.5 |
| RPMI 8226 | Lymph Leukemia | 6.5 | 0.0 | 3.6 | 0.0 |
| HS-578T | hu Ductal Carcinoma | 6.8 | 2.3 | 2.8 | 2.3 |
| IGR-OV1 | hu R Ovary Cysto Adenocarcinoma | 7.0 | 2.6 | 1.9 | 1.0 |
| BT-549 | hu Lymph Node Infil. Ductal Carcinoma | 7.2 | 2.1 | 4.8 | 3.3 |
| EKVX | hu Lung Adenocarcinoma | 7.2 | 4.2 | 7.7 | 7.3 |
| CAKI-1 | hu Renal Adenocarcinoma | 7.4 | 1.8 | 2.8 | 1.0 |
| Lewis Lung | hu Lung Carcinoma | 8.6 | 7.2 | 6.4 | 3.4 |
| 435 | Breast adenocarcinoma | 8.6 | 2.7 | 6.1 | 1.3 |
| NCI-H522 | hu Lung Adnocarcinoma | 9.1 | 3.7 | 5.1 | 1.7 |
| A549 | hu Lung Adenocarcinoma | 9.6 | 3.5 | 4.4 | 1.3 |
| ACHN | hu Renal Carcinoma | 9.6 | 2.9 | 8.0 | 3.1 |
| 231 | Breast adenocarcinoma | 9.6 | 2.6 | 3.4 | 1.1 |
| OVCAR-4 | hu Adenocarcinoma | 9.9 | 2.7 | 6.1 | 1.3 |
| SN12C | hu Renal Carcinoma | 10.6 | 3.5 | 6.7 | 6.4 |
| NCI-H23 | hu Lung Adenocarcinoma | 10.8 | 6.6 | 8.0 | 8.7 |
| MX-1 | hu Breast Mammary Carcinoma | 10.8 | 3.1 | 8.5 | 3.8 |
| A704 | hu Renal Adenocarcinoma | 10.9 | 1.8 | 4.5 | 1.2 |
| COLON 26 | Carcinoma | 11.3 | 2.3 | 8.9 | 2.2 |
| HOP 62 | hu Lung Adenocarcinoma | 12.0 | 0.9 | 4.1 | 0.2 |
| LOVO | hu Colon Adenocarcinoma | 12.6 | 5.4 | 8.7 | 3.8 |
| MOLT4 | Lymph Leukemia | 12.7 | 0.0 | 7.3 | 0.0 |
| SHP-77 | hu Lung Small Cell Carcinoma | 12.8 | 5.9 | 6.6 | 2.7 |
| HCT-116 | hu Colon Carcinoma | 14.1 | 4.4 | 12.4 | 9.5 |
| HOP 18 | hu Lung Large Cell | 16.6 | 8.1 | 10.3 | 3.1 |
| A2780 | hu Ovary Adenocarcinoma | 20.7 | 2.8 | 7.5 | 1.0 |
| PC-3 | hu Prostate Carcinoma | 23.2 | 8.5 | 44.2 | 13.2 |
| SR | Leukemia | 24.4 | 0.0 | 20.9 | 0.0 |
| CHA-59 | hu Bone Osteosarcoma | 24.7 | 9.7 | 8.2 | 2.1 |
| PAN 02 | Pancreatic Ductal Carcinoma | 25.8 | 7.0 | 9.3 | 2.2 |
| MCF 7 | Breast adenocarcinoma | 26.7 | 19.8 | 11.1 | 5.8 |
| A498 | hu Renal Carcinoma | 28.5 | 12.4 | 35.3 | 33.4 |
| NCI-H460 | hu Lung Large Cell Carcinoma | 30.3 | 5.6 | 11.6 | 5.8 |
| CCRF-CEM | Lymph Leukemia | 46.2 | 1.8 | 41.3 | 2.0 |
| Median | | 7.4 | 1.8 | 2.8 | 1.0 |
| HEK 293 | Kidney | 20.2 | 20.1 | 13.6 | 4.5 |

### Example 2

Seven matched pairs of tumor cell lines (one MBD high-uptake and one MBD low-uptake line for each tissue) were selected for further study. Of these, six pairs (all except the leukemia lines) were selected for gene array analysis.

TABLE 3

| TISSUE | HIGH-UPTAKE | LOW-UPTAKE |
|---|---|---|
| Prostate | PC-3 | DU-145 |
| Colon | HT-29 | HCT-15 |
| Lung | NCI-H23 | HOP-62 |
| Kidney | A498 | UO-31 |
| Ovary | OVCAR-8 | OVCAR-5 |
| Breast | MCF-7 | HS-578T |
| Leukemia | CCRF-CEM | K562 |

Total RNA was isolated using standard RNA purification protocols (Nucleospin RNA II). The RNA was quantified by photometrical measurement and the integrity checked by the Bioanalyzer 2100 system (Agilent Technologies, Palo Alto, Calif.). Based on electropherogram profiles, the peak areas of 28S and 18S RNA were determined and the ratio of 28S/18S was calculated. In all samples this value was greater than 1.5, indicating qualitative integrity of the RNAs. 1 μg total RNA was used for linear amplification (PIQOR™ Instruction Manual). Amplified RNA (aRNAs) were subsequently checked with the Bioanalyzer 2100 system. Samples yielded in every case >20 μg aRNA and showed a Gaussian-like distribution of the aRNA transcript lengths as expected (average transcript length 1.5 kB). This indicates successful amplification of the total RNA samples and good quality of the obtained aRNAs. All aRNAs were used for fluorescent label in PIQOR™ (Parallel Identification and quantification of RNAs) cDNA microarrays (Memorec Biotec GmbH, Cologne, Germany). cDNA microarray production, hybridization and evaluation were carried out as previously described [Bosio, A., Knorr, C., Janssen, U., Gebel, S., Haussmann, H. J., Muller, T., 2002. Kinetics of gene expression profiling in Swiss 3T3 cells exposed to aqueous extracts of cigarette smoke. Carcinogenesis 23, 741-748.]. Samples were labeled with FluoroLink™ Cy3/Cy5-dCTP (Amersham Pharmacia Biotech, Freiburg, Germany). 1 μg of amplified RNA for validation experiments were labeled and hybridized. All hybridizations were performed in quadruplicate. Quality controls, external controls and hybridization procedures and parameters were performed according to the manufacturer's instructions and comply to the MIAME standards. The Cy3 (sample) and Cy5 (reference) fluorescent labeled probes were hybridized on customized PIQOR™ Microarrays and subjected to overnight hybridization using a hybridization station. The arrays are designed to query genes previously implicated in processes relevant to cancer. These include 110 transcription factors, 153 extracellular matrix-related, 207 enzymes, 120 cell-cycle-related, 171 ligands/surface markers, and 368 signal transduction genes. Equal amounts of aRNA from the 12 respective cell lines were pooled and served as a reference against which each of the individual cell lines were hybridized.

Correlation analysis was carried out to identify those genes that might be implicated in the cellular physiological state most permissive for MBD-mediated uptake. Briefly, genes were sorted based on the -fold change in expression (up or down) when pairwise comparison of the selected high and low MBD-mediated uptake lines was performed by tissue. Based on an average of these -fold changes across all pairs, approximately the top (up-regulated) and bottom (down-regulated) 3% of the gene list was selected for further analysis. The functional distribution of genes in these two groups is highly non-random, as shown in Table 4.

TABLE 4

| | | HIGH vs LOW | |
|---|---|---|---|
| | % | MBD UPTAKE | |
| | ARRAY | UP-REG | DN-REG |
| GENE CATEGORY | (n = 1129) | (n = 32) | (n = 32) |
| TRANSCRIPTION FACTORS | 9.7 | 40.6 | 0 |
| INTRACELLULAR PROTEINS | 18.3 | 25.0 | 0 |
| SIGNAL TRANSDUCTION (I) | 32.6 | 9.4 | 0 |
| CELL-CYCLE, DNA REPAIR | 10.6 | 0 | 0 |
| ECM-RELATED | 13.6 | 3.1 | 68.8 |
| SURFACE MARKERS/LIGANDS | 15.2 | 9.4 | 31.2 |

There is a notable difference in the functional distribution of up- and down-regulated genes. The former primarily include transcription factors and other select intracellular proteins whereas the latter are exclusively extracellular. Using correlation of expression patterns across all cell lines to further sort the subsets of up- and down-regulated genes, it is possible to identify 2-3 major groupings in each set. Up-regulated genes include GDF15, SRC, ATF3, HSPF3, FAPP2, PSMB9, PSMB10, c-JUN, JUN-B, HSPA1A, HSPA6, NFKB2, IRF1, WDR9A, MAZ, NSG-X, KIAA1856, BRF2, COL9A3, TPD52, TAX40, PTPN3, CREM, HCA58, TCFL5, CEBPB, IL6R and ABCP2. It is remarkable that at least one third of these genes have been previously associated with cellular responses to stress (e.g. GDF15, ATF3, HSPF3, PSMB9, PSMB10, c-JUN, JUN-B, HSPA1A, HSPA6, NFKB2, IRF1). Down-regulated genes include CTGF, LAMA4, LAMB3, IL6, IL1B, UPA, MMP2, LOX, SPARC, FBN1, LUM, PAI1, TGFB2, URB, TSP1, CSPG2, DCN, ITGA5, TKT, CAV1, CAV2, COL1A1, COL4A1, COL4A2, COL5A1, COL5A2, COL6A2, COL6A3, COL7A1, COL8A1, and IL7R.

The patterns of up- or down-regulation of the following genes (shown in Table 5) serve as illustrations. Table 3 shows the fold expression difference in pairwise comparisons.

TABLE 5

| GENE | Prostate | Colon | Lung | Kidney | Breast |
|---|---|---|---|---|---|
| GDF-15 | 104.0 | 8.3 | 15.0 | 17.7 | 2.8 |
| IRF1 | 7.2 | 7.3 | 1.1 | 3.2 | 1.3 |
| HSP1A1 | 2.4 | 1.3 | 3.8 | 3.7 | 10.1 |
| JUNB | 9.0 | 0.9 | 6.1 | 3.2 | 10.0 |
| TGFB2 | 0.24 | 0.85 | 0.08 | 0.71 | 0.07 |
| IL6 | 1.05 | 0.67 | 0.26 | 0.21 | 0.04 |
| SPARC | 9.67 | 0.67 | 0.02 | 0.23 | 0.00 |

### Example 3

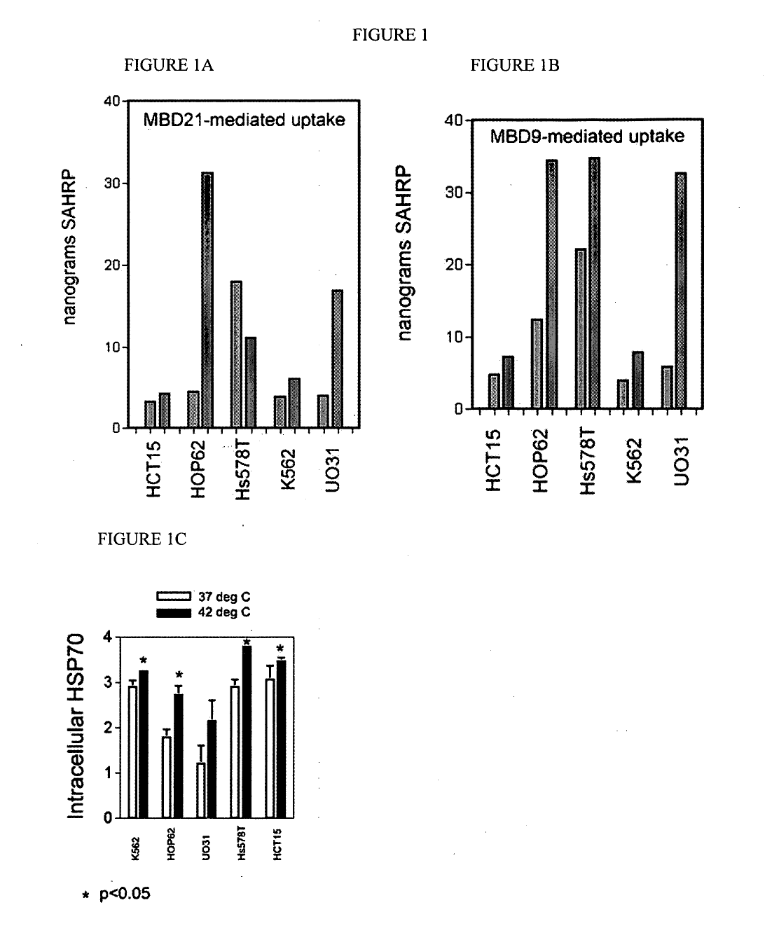

Low-uptake lines HCT-15, HOP-62, Hs578T, K562 and UO31 were heat-shocked at 42 degrees for 1 hour. HSP70 was induced by this treatment (FIG. 1C). Uptake of MBD-tagged peroxidase was measured in extracts from these cells (red bars, right) and from control cells at 37 degrees. Significantly higher uptake was seen in all cell lines upon heat shock, and this uptake was not due to increased permeability of cells as SAHRP control sample uptake was undetectable in all cases. Cells were grown in RPMI 1640 media+10% FBS+10 μm ferrous chloride until 85-90% confluency. They were trypsinized and removed from the plates. Cells were resuspended in the same media in 15 ml tubes and incubated at 42 degrees Celsius for one hour. There was a set of controls at 37 degrees Celsius for each cell line. Then 10 ul of each peptide complex was added to each tube (in duplicate) and incubated at 37 degrees Celsius for 20 minutes. After 20 minutes, the media was removed from the plates and the cells were washed with 1×PBS plus 1% calf serum twice. Extracts were made using NEPER Kit (Pierce Technology) and were assayed using the ELISA protocol for horseradish peroxidase. The cell extracts were prepared according to protocols provided with the nuclear extraction kits. Results are shown in FIGS. 1A and 1B. They show that heat shock increases uptake of MBD-mobilized SA-HRP.

### Example 4

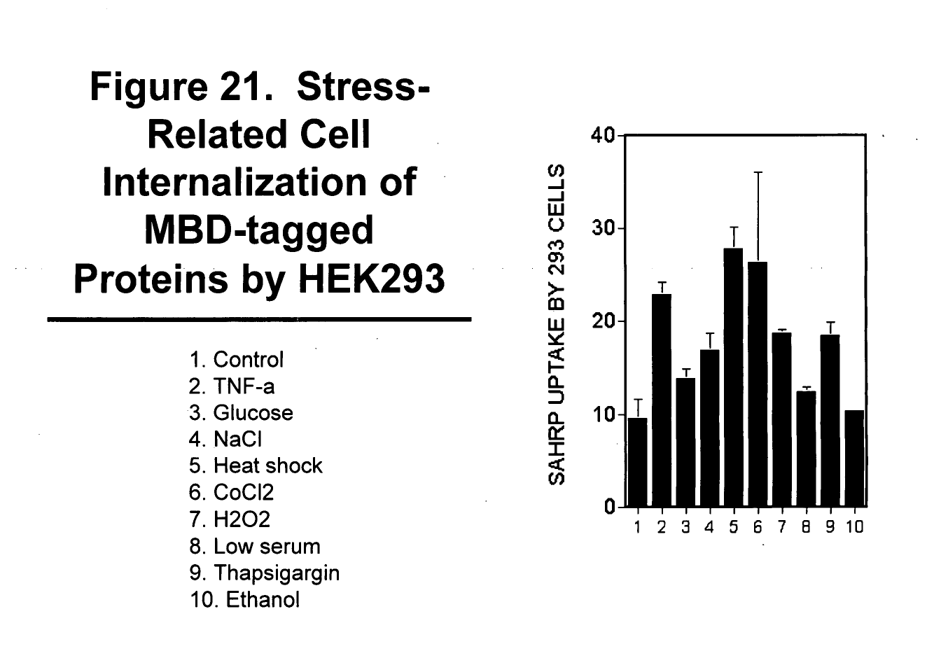

HEK293 cellular uptake of MBD9::SAHRP is stimulated by pre-treatment with stressors. Peroxidase activity was measured 20 minutes after addition of 100 ng/ml of MBD::SAHRP protein to the cell culture medium, as described in Example 1. All pretreatments were for 20 hours except for sample 5. The results of this experiment are shown in FIG. 21.

Sample Key: (1) 293 control (2) 293+30 ng/ml TNF-a (3) 293+25 mM D-glucose (4) 293+700 mM NaCl (5) 293+42 deg C., 1 hour (6) 293+200 uM Cobalt chloride (7) 293+200 uM hydrogen peroxide (8) 293+low (1%) serum (9) 293+300 nM thapsigargin (10) 293+100 uM ethanol.

### Example 5

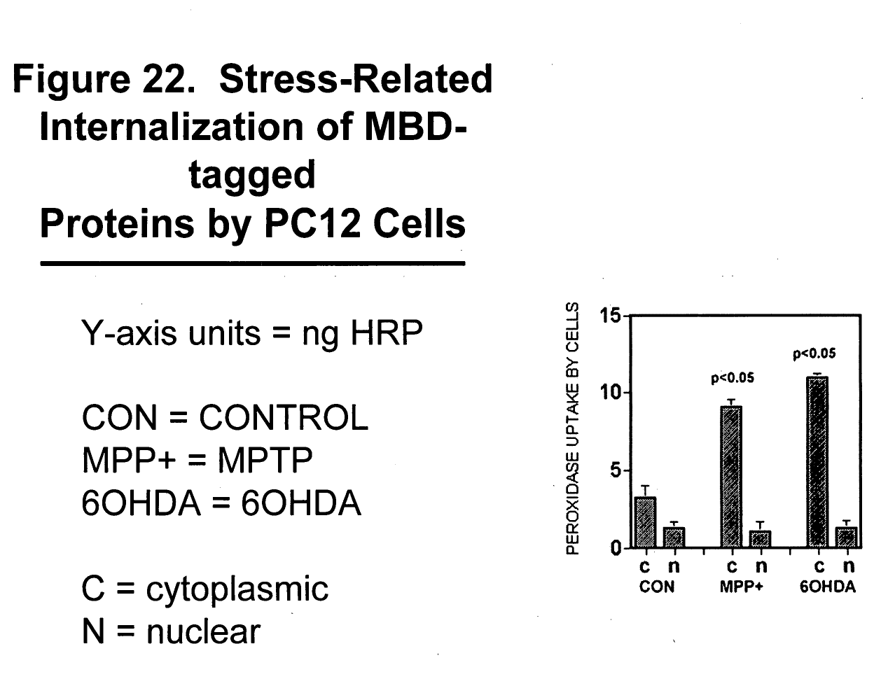

MBD-mediated protein mobilization into PC12 cells is stimulated by stressors used in models of PD. 6-OHDA or MPP+treatment of PC 12 cells dramatically stimulates uptake of MBD-mobilized horseradish peroxidase. PC12 cells cultured in RPMI 1640+FBS were pretreated with MPTP or 6-OHDA. Uptake of exogenously added MBD::SAHRP (100 ng/ml) was measured in nuclear and cytoplasmic extracts 20 minutes after addition of the protein to the cell culture medium. The results are shown in FIG. 22. They confirm that experimental stressors routinely used in experimental models of PD also stimulate cellular uptake of MBD-tagged proteins in PC12 cells.

### Example 6

Combinations of stressors can have novel effects on cellular uptake of MBD-tagged proteins in HEK293 cells and can be modulated by IGF-I. HEK293 cells were grown in 1% serum (nutritional stress) and peroxidase activity was measured 20 minutes after addition of 100 ng/ml of MBD::SAHRP protein to the cell culture medium, as described in Example 1. All pretreatments with growth factors IGF-I or EGF (100 ng/ml) were for 2 hours, followed by the indicated stress treatment (heat shock at 42 degrees Celsius for 60 minutes or 200 uM Cobalt Chloride for 60 minutes to simulate anoxia). Uptake was measured at the end of the stress treatment. The results are shown in the table below (p values shown are relative to the control without growth factor treatment in each group; only significant p values are shown):

| Secondary Stressor | Growth Factor | Uptake of MBD::SAHRP (ng) |
|---|---|---|
| NONE | NONE | 20.10 ± 1.22 |
| HEAT SHOCK | NONE | 4.71 ± 0.80 |
| | | (p < 0.01) |
| HEAT SHOCK | +IGF-I | 2.54 ± 0.54 |
| | | (p = 0.023) |
| HEAT SHOCK | +EGF | 6.00 ± 0.56 |
| COBALT (ANOXIA) | NONE | 20.91 ± 1.22 |
| COBALT (ANOXIA) | +IGF-I | 25.29 ± 0.57 |
| | | (p = 0.013) |
| COBALT (ANOXIA) | +EGF | 25.59 ± 1.0 |
| | | (p = 0.008) |

### Example 7

Combinations of stressors can have novel effects on cellular uptake of MBD-tagged proteins in MCF-7 cells and can be modulated by IGF-I. MCF-7 cells were grown in 1% serum (nutritional stress) and peroxidase activity was measured 20 minutes after addition of 100 ng/ml of MBD::SAHRP protein to the cell culture medium, as described in Example 1. All pretreatments with growth factors IGF-I or EGF (100 ng/ml) were for 2 hours, followed by the indicated stress treatment (heat shock at 42 degrees Celsius for 60 minutes or 200 uM Cobalt Chloride for 60 minutes to simulate anoxia). Uptake was measured at the end of the stress treatment. The results are shown in the table below (p values shown are relative to the control without growth factor treatment in each group; only significant p values are shown):

| Secondary Stressor | Growth Factor | Uptake of MBD::SAHRP (ng) |
|---|---|---|
| NONE | NONE | 20.63 ± 0.87 |
| HEAT SHOCK | NONE | 1.67 ± 1.11 |
| | | (p < 0.01) |
| HEAT SHOCK | +IGF-I | 1.19 ± 0.21 |
| HEAT SHOCK | +EGF | 2.11 ± 1.50 |
| COBALT (ANOXIA) | NONE | 22.83 ± 0.73 |
| | | (p = 0.030) |
| COBALT (ANOXIA) | +IGF-I | 20.71 ± 1.01 |
| | | (p = 0.048) |
| COBALT (ANOXIA) | +EGF | 23.91 ± 0.72 |

### Example 8

Peptide Bio-KGF binds shRNA: Bio-KGF peptide was synthesized by Genemed Synthesis, Inc. (S. San Francisco, Calif.) as a 40-mer containing an MBD sequence and an RNA-hairpin binding domain from the N-terminus of bacteriophage lambda N protein: Bio-KGF: (“N”-terminal biotin) . . . KGF YKK KQC RPS KGR KRG FCW AQT RRR ERR AEK QAQ WKA A . . . (“C” terminus)

An shRNA designed to silence the human beclin gene was designed to include a hairpin sequence corresponding to the NutR box of bacteriophage lambda mRNA (the binding target for the Bio-KGF peptide) and was amplified using the Silencer™ siRNA

Construction Kit (Ambion) using conditions specified by the manufacturer. The sequence of the DNA oligonucleotide used for the kit transcription reaction was:

T7BECR:
5′...AG TTT GGC ACA ATC AAT AAC TTTTTC AGT TAT TGA
TTG TGC CAA ACT CCTGTCTC...3′

As a vector control for in vivo confirmation of siRNA efficacy, the following oligonucleotides were designed for cloning into the pGSU6 vector (BamHI-EcoRI)

BECF:
5′...GAT CGG CAG TTT GGC ACA ATC AAT AAC TGAAAA
AGT TAT TGA TTG TGC CAA ACT GTT TTT TGG AAG...3′
BECR:
5′...AAT TCT TCC AAA AAA CAG TTT GGC ACA ATC AAT
AAC TTTTTC AGT TAT TGA TTG TGC CAA ACT GCG...3′

Various molar excess amounts of Bio-KGF (ranging from 63 pg to 2 ug per well; similar results were obtained across this range) were attached to a Ni-NTA plate (Qiagen Inc., Carlsbad, Calif.) for 1 hour and blocked overnight with 3% BSA at 4 degrees C. in the refrigerator, and washed with PBS/Tween and TE buffers. RNA dilutions were added in TE buffer, incubated for 30 min on shaker, then for 30 min on bench at room temperature. After one wash with TE buffer, Ribogreen reagent (Ribogreen RNA Quantitation Reagent and Kit from Molecular Probes/Invitrogen) was added to the wells, incubated 5 minutes, and fluorescence was read on a fluorescent plate reader. The results are listed in the following table (each number is a mean of eight readings:

| ng shRNA per well | Ribogreen Fluorescence |
|---|---|
| 88 | 81819 ± 24656 |
| 44 | 42053 ± 12769 |
| 22 | 11924 ± 3650 |
| 11 | 6016 ± 2977 |
| 5.5 | 2058 ± 781 |
| 2.7 | 853 ± 600 |

The Bio-KGF peptide binds the shRNA containing the lambda nutR hairpin loop.

Although the foregoing invention has been described in some detail by way of illustration and example for purposes of clarity of understanding, the descriptions and examples should not be construed as limiting the scope of the invention.

1. A method for delivering an MBD peptide-linked agent into live cells, said method comprising contacting said MBD peptide-linked agent to live cells that are under a condition of cellular stress, whereby said contact results in cellular uptake of said MBD-peptide-linked agent.

2. The method of claim 1, wherein the condition of cellular stress is selected from the group consisting of thermal, immunological, cytokine, oxidative, metabolic, anoxic, endoplasmic reticulum, protein unfolding, nutritional, chemical, mechanical, osmotic and glycemic stress.

3. The method of claim 1, further comprising the steps of comparing levels of gene expression of one or more of the genes shown in FIG. 7 in said live cells under the condition of cellular stress to levels of gene expression in the same type of live cells not under the condition of cellular stress; and selecting live cells that have at least three of the genes shown in FIG. 7 upregulated at least 1.5-fold under the condition of cellular stress for delivering the MBD peptide-linked agent into the live cells.

4. The method of claim 1, wherein the agent is a diagnostic agent or a therapeutic agent.

5. The method of claim 4, wherein the agent is a protein or a peptide.

6. The method of claim 4, wherein the agent is a nucleic acid.

7. The method of claim 4, wherein the agent is a small molecule.

8. The method of claim 1, wherein the MBD peptide comprises the amino acid sequence QCRPSKGRKRGFCW.

9. The method of claim 1, wherein the MBD peptide comprises the amino acid sequence QCRPSKGRKRGFCW and a caveolin consensus binding sequence.

10. The method of claim 1, wherein the MBD peptide comprises the amino acid sequence QCRPSKGRKRGFCWAVDKYG or KKGFYKKKQCRPSKGRKRGFCWAVDKYG.

11. A method for obtaining diagnostic information from live cells comprising the steps of:
(a) administering an MBD peptide-linked agent to live cells that are under a condition of cellular stress;
(b) delivering said MBD peptide-linked agent into said live cells, whereby said agent creates a diagnostic readout that can be measured; and
(c) measuring the diagnostic readout.

12. The method of claim 11, wherein the diagnostic readout is selected from the group consisting of enzymatic, colorimetric, and fluorimetric readout.

13. A method for modifying in a disease process or a cellular process, said method comprising the steps of:
(a) administering an MBD peptide-linked agent to live cells that are under a condition of cellular stress, wherein said agent is capable of modifying the disease process or the cellular process within said live cells; and
(b) delivering said MBD peptide-linked agent into said live cells, whereby said disease process or said cellular process in said live cells is modified.

14. The method of claim 13, wherein said disease process is selected from the group consisting of neurodegenerative, cancer, autoimmune, inflammatory, cardiovascular, diabetes, osteoporosis and ophthalmic diseases.

15. The method of claim 13, wherein said cellular process is selected from the group consisting of transcriptional, translational, protein folding, protein degradation and protein phosphorylation events.
